(19) United States
(12) Patent Application Publication
(10) Pub. No.: US 2008/0275076 A1
(43) Pub. Date: Nov. 6, 2008

(54) Pharmaceutical Compositions Comprising Sirolimus and/or an Analogue Thereof

(76) Inventors: Per Holm, Vanlose (DK); Tomas Norling, Lyngby (DK)

Correspondence Address: CHERYL H AGRIS PHD, PO BOX 806, PELHAM, NY, 10803, US

(21) Appl. No.: 11/885,992

(22) PCT Filed: Mar. 8, 2006
(86) PCT No.: PCT/DK2006/000135

§ 371 Date: May 8, 2008

(30) Foreign Application Priority Data
Mar. 8, 2005 (DK) PA 2005 00344

(51) Int. Cl. A61K 31/4353; A61P 37/06

(52) U.S. Cl. 514/291

(57) ABSTRACT

The present invention relates to pharmaceutical compositions in particulate form or in solid dosage forms comprising sirolimus (rapamycin) and/or derivatives and/or analogues thereof. Compositions of the invention exhibit an acceptable bioavailability of sirolimus and/or a derivative and/or an analogue thereof. The pharmaceutical compositions of the invention are designed to release sirolimus in a controlled manner so that the plasma levels stays within the narrow therapeutic window that exist for this class of substances. An extended release profile, where the peak concentration has been reduced without loosing significant bioavailability, together with less variable absorption, is expected to improve the safety/efficacy ratio of the drug. Furthermore, compositions according to the invention provide for a significant reduced food effect and a delayed release of sirolimus is expected to reduce the number of gastro-intestinal related side effects.

The present invention relates to pharmaceutical compositions in particulate form or in solid dosage forms comprising sirolimus (rapamycin) and/or derivatives and/or analogues thereof. Compositions of the invention exhibit markedly decreased variability of sirolimus and/or a derivative and/or an analogue thereof compared to commercially available solid dosage forms containing sirolimus. The pharmaceutical compositions of the invention are designed to release sirolimus in a controlled manner e.g. in an extended manner so that the plasma levels stays within the narrow therapeutic window that exist for this class of substances. An extended release profile, where the peak concentration has been reduced without loosing significant bioavailability, together with less variable absorption, is expected to improve the safety/efficacy ratio of the drug. Furthermore, compositions according to the invention are contemplated to provide for a significant reduced food effect and a delayed release of sirolimus is expected to reduce the number of gastro-intestinal related side effects.

In particular the invention relates to solid pharmaceutical compositions comprising sirolimus and/or a derivative and/or an analogue thereof dissolved or dispersed in a vehicle suitable for oral administration.

### BACKGROUND OF THE INVENTION

Sirolimus is an immunosuppressive agent. It is a macrocyclic lactone produced by Streptomyces hygroscopicus. The chemical name is (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[1S,3R,4R)-4-hydroxy-3-methoxycyclohexyl]-1-methylethyl]-10,21-dimethoxy-6,8,12,14,20,26-hexamethyl-23,27-epoxy-3-H-pyrido[2,1-c][1,4]-oxaazacyclohentriacontine-1,5,11,28,29(4H,6H,31H)-pentone. Sirolimus (also denoted rapamycin) has the tricyclic structure shown below.

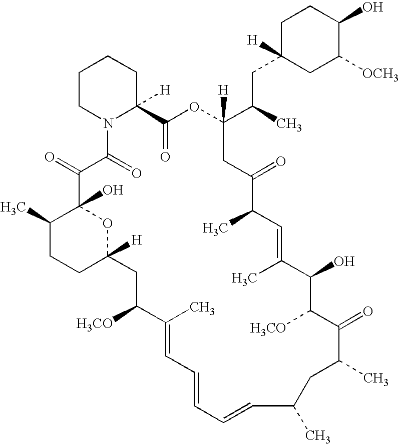

C₅₁H₇₉NO₁₃, molecular weight 914.2.

Sirolimus is a white to off-white powder and is insoluble in water, but freely soluble in benzyl alcohol, chloroform, acetone and acetonitrile. Within the scope of the present invention is sirolimus in any physical form (crystals, amorphous powder, any possible polymorphs, any possible solvates include the hydrate, anhydrate, complexes thereof etc.). Included is also any analogue of sirolimus, pharmaceutically acceptable salts, solvates, complexes and prodrugs thereof.

The preparation of rapamycin is described in U.S. Pat. No. 3,929,992 which is hereby incorporated by reference.

Sirolimus is a macrolide compound with useful immunosuppressive activity, antimicrobial activity and other pharmacological activities and is of value for the treatment or prevention of rejection reactions by transplantation of organs or tissues, graft versus host diseases, autoimmune diseases and infectious diseases.

Sirolimus inhibits T-lymphocyte activation and proliferation that occurs in response to antigenic and cytokine stimulation by a mechanism that is distinct from that of other immunosuppressants. Sirolimus also inhibits antibody production. In cells, sirolimus binds to the immulophilin, FK Binding Protein-12 to generate an immunosuppressive complex. This complex has no effect on calcineurin activity. The complex binds to and inhibits the activation of the mammalian “target of rapamycin” (nTOR), a key regulatory kinase. This inhibition suppresses cytokine-driven T-cell proliferation, inhibiting the progression from the G₁ to the S phase of the cell cycle.

Studies in experimental models show that sirolimus prolongs allograft (kidney, heart, skin, islet, small bowel, pancreatico-duodenal, and bone marrow) survival in e.g. mice, rats, pigs, and primates. Sirolimus reverses acute rejection of heart and kidney allografts and prolongs the graft survival in rats.

In rodent models of autoimmune diseases, sirolimus suppresses immune-mediated events associated with systemic lupus erythematosus, collagen-induced arthritis, autoimmune type I diabetes, autoimmune myocarditis, experimental allergic encephalomyelitis, graft-versus-host disease, and autoimmune uveorentinitis.

A commercially available sirolimus-containing product is Rapamune®. Rapamune® is indicated for the prophylaxis of organ rejection in patients receiving renal transplants. It is recommended that Rapamune® be used in a regiment with cyclosporine and corticosteroids.

Usually sirolimus is administered orally and is therefore absorbed from the gastrointestinal tract. It has been observed that the absorption is influenced by the simultaneous ingestion of food. Thus, the extent of sirolimus absorption (AUC) was greatest when it was taken orally together with high-fat meal conditions. However, compared to fasting, a decrease in the peak blood sirolimus concentration (Cₘₐₓ), an increase in the time-to-peak concentration (tₘₐₓ) and an overall increase in total exposure (AUC) were observed. Accordingly, it is recommended that Rapamune® is taken consistently with or without food.

In general, it is known that the absorption and bioavailability of a therapeutically active substance can be affected by a variety of factors when administered orally. Such factors include the presence of food in the gastrointestinal tract and, in general, the gastric residence time of a drug substance is significantly longer in the presence of food than in the fasted state. If the bioavailability of a drug substance is affected beyond a certain point due to the presence of food in the gastrointestinal tract, the drug substance is said to exhibit a food effect. Food effects are important because there is a risk associated with administering the drug substance to a patient who has eaten recently. The risk derives from the potential that absorption into the bloodstream may be adversely affected to the point that the patient risks insufficient absorption to remedy the condition for which the drug was administered.

Absorption of sirolimus from the gastrointestinal tract after oral administration is rapid with a mean time-to-peak concentration (tₘₐₓ) of approximately 1 hour after a single dose in healthy subjects and approximately 2 hours after multiple oral doses in renal transplant recipients. The systemic availability of sirolimus was estimated to be approximately 14% after oral administration of Rapamune® oral solution. The mean bioavailability of sirolimus after administration of Rapamune® tablets is about 27% higher relative to the oral solution.

Sirolimus is a substrate for both cytochrome P450 IIIA4 (CYP3A4) and P-glycoprotein. Sirolimus is extensively metabolized by O-demethylation and/or hydroxylation. Seven major metabolites, including hydroxy, demethyl and hydroxydemethyl are identifiable in whole blood. Glucuronide and sulfate conjugates are not present in any of the biologic matrices. Sirolimus is the major component in human whole blood and contributes to more than 90% or the immunosuppressive activity.

Furthermore, oral administration of sirolimus is associated with side effects including hypercholesterolemia, hyperlipemia hypertension and rash.

Sirolimus is extensively metabolized by the CYP3A4 isoenzyme in the gut wall and liver. Therefore, absorption and the subsequent elimination of systemically absorbed sirolimus may be influenced by drugs that affect this isoenzyme. Inhibitors of CYP3A4 may decrease the metabolism of sirolimus and increase sirolimus levels, while inducers of CYP3A4 may increase the metabolism of sirolimus and decrease sirolimus levels. Accordingly, sirolimus may be administered together with one or more CYP3A4 inhibitors in order to improve the overall bioavailability.

For oral administration, sirolimus is currently formulated and marketed as an oral solution containing 1 mg/ml sirolimus. Rapamune® is also available as a white, triangular-shaped tablet containing 1 mg sirolimus, and as a yellow to beige triangular-shaped tablet containing 2 mg sirolimus. Rapamune® oral solutions contains sirolimus and as inactive ingredients Phosal 50 PG® (phosphatidylcholine, propylene glycol, mono- and disaccharides, ethanol, soy fatty acids, and ascorbyl palmitate) and polysorbate 80. Rapamune® tablets contain—apart from sirolimus—sucrose, lactose, polyethylene glycol 8000, calcium sulfate, microcrystalline cellulose, pharmaceutical glaze, talc, titanium dioxide, magnesium stearate, povidone, poloxamer 188, polyethylene glycol 20,000, glycerol monooleate, carnauba wax etc.

Rapamune® is intended for administration orally once daily. However, shortly after transplantation a loading dose of 3 times the maintenance dose should be given. A daily maintenance dose of 2 mg is recommended for use in renal transplant patient.

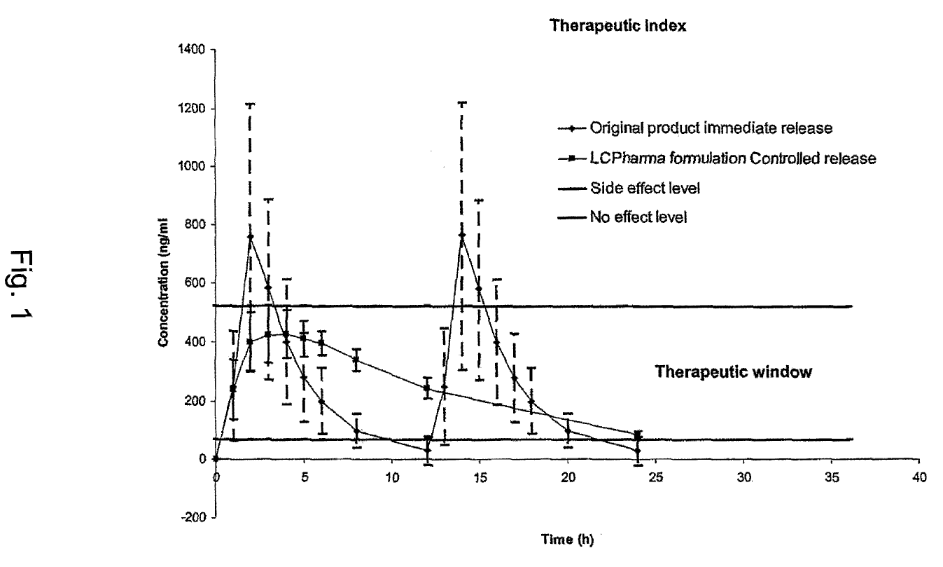

There remains a need for new pharmaceutical compositions comprising sirolimus exhibiting, a reproducible, controlled release of the drug with plasma levels which can stay within the narrow therapeutic window (see FIG. 1) for an extended period of time, without loosing significant bioavailability. A reduction in food effect, to help ensure, that plasma levels stays within the desired values.

Further, pharmaceutical compositions comprising sirolimus and exhibiting a reproducible (i.e. less variable compared to that of Rapamunef®) extended release profile of this compound may allow a reduction in the dosage units taken by a patient, e.g. down to a single dose daily, and may also reduce or negate the need for food to be takes simultaneously with the dosage form thereby allowing patients more freedom on when the drug is taken. Furthermore, it is contemplated that fluctuations in the plasma concentration versus time profile may be significantly reduced.

Delaying the release of sirolimus to the distal part of duodenum is expected to reduce the drug related gastro-intestinal related side effects and the relatively high degree of metabolism in the proximal part of the gastrointestinal tract (CyP3A4 and P-glycoprotein mediated metabolism). Owing to the compositions/technology, this is done without loosing systemic bioavailability.

Whenever the term sirolimus is used in the present context it is intended to denote sirolimus in any form (e.g. crystalline, polymorphous or amorphous form, solvate, hydrate, anhydrate etc) as well as analogues, derivatives or prodrugs thereof.

### DESCRIPTION OF THE INVENTION

As mentioned above, there is a need for developing pharmaceutical sirolimus-containing compositions notably for oral use that lead to an improved treatment of conditions with sirolimus. An improved release profile, which can ensure significant lower Cₘₐₓ, but still good bioavailability, as well as an extended release of drug staying within the therapeutic plasma levels for up to 24 hours after administration. A further therapeutic improvement of the invention is the reduction in food effect, which together with the improved absorption should give more reproducible plasma levels. The therapeutic improvements of the invention will clearly improve the ratio between side effects and efficacy. Another way of obtaining an improved treatment of conditions where sirolimus is indicated is by balancing the release of sirolimus to the gastro-intestinal tract in such a manner that an enhanced plasma concentration of sirolimus is obtained initially or delayed with respect to the time of administration.

The present invention provides a pharmaceutical composition comprising sirolimus together with one or more pharmaceutically acceptable excipient, wherein the composition upon oral administration to a mammal releases sirolimus in a controlled manner and reduces inter- and/or intra-individual variations compared to those of Rapamune® administered under the same conditions.

More specifically, a pharmaceutical composition according to the invention comprises a vehicle that has a melting point of 80° C. or less and wherein the solubility of sirolimus is at least 0.5% w/w at a temperature corresponding to the melting point of the vehicle, wherein the coefficient of variation (CV) of Cₘₐₓ and/or of AUC_{inf} after administration to six healthy fasting subjects or four healthy dogs is at the most 30%.

In specific embodiments and as it appears from the examples herein, a pharmaceutical composition according to the invention has a CV of AUC_{inf} of at the most 25%. Accordingly, the CV is markedly reduced compared to what has been obtained by administration of a commercially available sirolimus-containing product, Rapamune® tablets under the same conditions (including dose, fasting, non-fasting, access to water, monitoring etc.). A measure for such an improvement is the ratio (CV_{Control}−CV)/CV_{Control}×100% which is at least 20% and the of CV is the CV of Cₘₐₓ and/or of AUC_{inf}, and CV_{Control} is determined under similar conditions as CV using Rapamune® tablets as control. In a specific embodiment the ratio is at least 25%. In these determinations the CV may be CV of Cₘₐₓ. In other embodiments, the ratio is at least 30% such as, e.g., at least 35%, at least 40%, at least 45% or at least 50%. In such determinations, the CV is CV of AUC_{inf}.

Under the heading “Vehicles” herein is given a variety of substances that alone or in combination may be used as vehicles. Some of the substances mentioned can only be used in combination with other substances due to the fact that these substances alone does not fulfil the criterial defined herein for qualifying as a vehicle. Just to mention one, HMPC cannot be used as a vehicle alone as its melting point is much higher than 80° C. In the following is given a selection of particularly suitable vehicles for use in the present invention, namely at least one of Rylo MD50, Gelucire 44/14, PEG such as PEG 6000, Poloxamer such as Poloxamer 188, Monomuls 90 L12 and Monomuls 90 35, and mixtures thereof.

In the following table is given the solubility of sirolimus in various vehicles (please note that the solubility is determined by visual inspection):

| | % |
|---|---|
| | Sirolimus |
| | dissolved |
| Vehicle | 70° C. |
| Rylo MD50 | 1.5 |
| Gelucire 44/14 | 2.5 |
| PEG 6000 | 1.5 |
| Poloxamer 188 | 1.0 |
| PEG 6000/Poloxamer 188 (70:30) | 2.4 |
| Monomuls 90 L12/Rylo MD50 (10:90) | 2.5 |
| Monomuls 90L12 | 3.9 |
| Monomuls 90 35 | 2.2 |

In a specific embodiment a pharmaceutical composition according to the invention is in solid form such as a solid dosage form including tablets.

Moreover in an embodiment aiming at obtaining a solid solution or dispersion of sirolimus in the vehicle, the concentration of sirolimus in the vehicle at the most corresponds to the solubility of sirolimus in the vehicle at 70° C.

In general, the concentration of sirolimus in the vehicle is at the most about 10% w/w such as at the most about 5% w/w, at the most about 4% w/w, at the most about 3% w/w, at the most about 2% w/w or at the most about 1% w/w.

As seen from the examples herein the preparation of the pharmaceutical composition normally involves a step, wherein sirolimus is dissolved in the vehicle at a temperature in a range of from about 50° C. to about 80° C.

A pharmaceutical composition according to the invention may contain any relevant amount of sirolimus. Normally, a solid dosage form of the invention comprises one or more multipla of 0.25 mg of sirolimus and/or from about 0.25 mg to about 5 mg of sirolimus. In specific embodiments a pharmaceutical composition according to the invention comprises a dose of 0.75 mg, 1 mg, 1.2 mg, 1.5 mg or 2 mg of sirolimus or from about 50% to about 80% of said dose.

With respect to the concentration of sirolimus in the composition, it is generally from about 0.05% to about 20% w/w such as, e.g., from about 0.05% to about 15% w/w, from about 0.05 to about 10% w/w, from about 0.1% to about 10% w/w,

In a preferred embodiment, the concentration of sirolimus in the composition is from about 0.05% to about 5% w/w, from about 0.1% to about 5% w/w, from about 0.1% to about 2.5% w/w, from about 0.5% to about 2.5% w/w, from about 1% to about 2.5% or 1% w/w or less.

The vehicle normally constitutes at the most 60% w/w of the composition and/or the vehicle at least constitute 20% w/w of the composition such as, e.g. at least about 30% w/w or at least about 40% w/w.

The present invention provides means for obtaining pharmaceutical compositions with a controlled release of sirolimus. As explained herein, a controlled release may be a release that enables a relatively fast onset of action (i.e. in which case it is not an object to smoothening the plasma concentration-time profile, but to obtain a fast appearance of sirolimus in the plasma in a therapeutically relevant concentration) or it may be a release that extend the duration of action. More particulars and details are given in the appended claims herein and specific embodiments of the present invention provide sirolimus-containing compositions designed for a relatively fast onset of action after administration to a subject. To this end the following applies:

T_{0.5h} is at least 50% of Tₘₐₓ such as, e.g., at least 60%, at least 65%, at least 70%, at least 75% or at least 80% of Tₘₐₓ and T_{0.5h} and Tₘₐₓ are determined as average values after administration to six healthy fasting subject or four healthy fasting dogs, and/or

T₁ₕ is at least 80% of Tₘₐₓ such as, e.g., at least 85%, at least 90%, at least 95% of Tₘₐₓ and T_{0.5h} and Tₘₐₓ are determined as average values after administration to six healthy fasting subject or four healthy fasting dogs, and/or

Tₘₐₓ is at the most 1.5 hours such as, e.g., 1.2 hours, 1.1 hours or 1 hours determined as an average of Tₘₐₓ after administration to six healthy fasting subjects, and/or

Tₘₐₓ is at the most 1.5 hours such as, e.g., 1.2 hours, 1.1 hours or 1 hours as determined as an average of Tₘₐₓ after administration to our healthy fasting dogs, and/or

Tₘₐₓ/T_{max, Control}×100% is at the most 70% such as, e.g., at the most 65%, at the most 60% or at the most 55%.

In a composition designed for fast onset of action it is preferably essentially that it does not contain HPMC such as is without any content of HPMC.

The present invention provides a pharmaceutical composition containing sirolimus, wherein the release of sirolimus is designed to avoid high peak concentrations and at the same time, the composition is designed so that the overall bioavailability is essentially maintained or increased (compared to commercially available sirolimus-containing products). Moreover, by delaying the release of sirolimus and at the same time provide a composition wherein sirolimus is at least partly on dissolved form, it is possible to obtain a significant absorption in the distal part of the gastrointestinal tract.

The present invention provides pharmaceutical compositions and solid dosage forms for improved treatment of conditions that respond to sirolimus therapy.

Within the scope of the present invention sirolimus may be in any physical form (crystals, amorphous powder, any possible polymorphs, any possible solvates including the hydrate, anhydrate, complexes thereof etc.). Included is also any analogue, derivative or active metabolite of sirolimus, pharmaceutically acceptable salts, solvates, complexes and prodrugs thereof.

Known indications of sirolimus are e.g.

i) treatment and prevention of organ or tissue allo- or xeno-transplant rejection, e.g. for the treatment of recipients of e.g. heart, lung, combined heart-lung, liver, kidney, bone marrow, small intestine, limb, muscle, nerve, intervertebral disc, trachea, myoblast, cartilage, pancreatic, skin or corneal transplants etc. It is also indicated for the prevention of graft-versus-host disease, such as following bone marrow transplantation,
ii) treatment and prevention of autoimmune disease and of inflammatory conditions, in particular inflammatory conditions with an etiology including an autoimmune component such as arthritis (for example rheumatoid arthritis, arthritis chronica progrediente and arthritis deformans) and rheumatic disease. Specific autoimmune diseases for which sirolimus may be employed include, autoimmune hematological disorders (including e.g. hemolytic anaemia, aplastic anaemia, pure red cell anaemia and idiopathic thrombocytopenia), systemic lupus erythematosus, Hashimoto's thyroiditis, multiple sclerosis, type I diabetes, polychondritis, sclerodoma, Wegener granulamatosis, dermatomyosistis, chronic active hepatitis, myasthenia gravis, psoriasis, Steven-Johnson syndrome, idiopathic sprue, autoimmune inflammatory bowel disease (including e.g. ulcerative colitis and Crohn's disease) endocrine opthalmopathy, Graves disease, sarcoidosis, multiple sclerosis, primary billiary cirrhosis, juvenile diabetes (diabetes mellitus Type I), uveitis (anterior and posterior), keratoconjunctivitis sicca and vernal keratoconjunctivitis, interstitial lung fibrosis, psoriatic arthritis, glomerulonephritis (with or without nephritic syndrome, e.g. including idiopathic nephritic syndrome or minimal change nephropathy) and juvenile dermatomyositis,
iii) treatment and prevention of asthma,
iv) treatment of multi-drug resistance (MDR). MDR is particularly problematic in cancer patients and AIDS patients. The compositions of the invention are therefore useful for enhancing the efficacy of other chemotherapeutic agents in the treatment and control of multi-drug resistant conditions such as multidrug resistant cancer or multidrug resistant AIDS,
v) treatment of proliferative disorders, e.g. tumors, hyperproliferative skin disorders and the like,
vi) treatment of fungal infections,
vii) treatment and prevention of inflammations, especially in potentiating the action of steroids,
viii) treatment and prevention of infection, especially infection by pathogens having Mip or Mip-like factors,
ix) treatment of an overdose of tacrolimus and other macrophilin binding immunosuppresants
x) infections caused by pathogenic microorganisms (e.g. Aspergillus fumigatus, Fusarium oxysporum, Trichophyton asteroides, etc.);
xi) inflammatory or hyperproliferative skin diseases or cutaneous manifestations of immunologically mediated diseases (e.g. psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, lupus erythematosus, acne, and alopecia greata);
xii) autoimmune diseases of the eye (e.g. keratoconjunctivitis, vernal conjunctivitis, uveitis associated with Behcet's disease, keratitis, herpetic keratitis, conical keratitis, corneal epithelial dystrophy, keratoleukoma, ocular premphigus, Mooren's ulcer, scleritis, Graves' opthalmopathy, Vogt-Koyanagi-Harada syndrome, keratoconjunctivitis sicca (dry eye), phlyctenule, iridocyclitis, sarcoidosis, endocrine opthalmopathy, etc.);
xiii) reversible obstructive airways diseases [asthma (e.g. bronchial asthma, allergic asthma, intrinsic asthma, extrinsic asthma, and dust asthma), particularly chronic or inveterate asthma (e.g. late asthma and airway hyper-responsiveness) bronchitis, etc.];
xiv) mucosal or vascular inflammations (e.g. gastric ulcer, ischemic or thrombotic vascular injury, ischemic bowel diseases, enteritis, necrotizing enterocolitis, intestinal damages associated with thermal burns, leukotriene B4-mediated diseases);
xv) intestinal inflammations/allergies (e.g. coeliac diseases, proctitis, eosinophilic gastroenteritis, mastocytosis, Crohn's disease and ulcerative colitis);
xvi) food-related allergic diseases with symptomatic manifestation remote from the gastrointestinal tract (e.g. migrain, rhinitis and eczema);
xvii) renal diseases (e.g. intestitial nephritis, Goodpasture's syndrome, hemolytic uremic syndrome, and diabetic nephropathy);
xviii) nervous diseases (e.g. multiple myositis, Guillain-Barre syndrome, Meniere's disease, multiple neuritis, solitary neuritis, cerebral infarction, Alzheimer's diseases Parkinson's diseases, amyotrophic lateral sclerosis (ALS) and radiculopathy);
xix) cerebral ischemic disease (e.g., head injury, hemorrhage in brain (e.g., subarachnoid hemorrhage, intracerebral hemorrhage), cerebral thrombosis, cerebral embolism, cardiac arrest, stroke, transient ischemic attack (TIA), hypertensive encephalopathy, cerebral infarction);
xx) endocrine diseases (e.g. hyperthyroidism, and Basedow's disease);
xxi) hematic diseases (e.g. pure red cell aplasia, aplastic anemia, hypoplastic anemia, idiopathic thrombocytopenic purpura, autoimmune hemolytic anemia, agranulocytosis, pernicious anemia, megaloblastic anemia, and anerythroplasia);
xxii) bone diseases (e.g. osteoporosis);
xxiii) respiratory diseases (e.g. sarcoidosis, pulmonary fibrosis, and idiopathic interstitial pneumonia);
xxiv) skin diseases (e.g. dermatomyositis, leukoderma vulgaris, ichthyosis vulgaris, photosensitivity, and cutaneous T-cell lymphoma);
xxv) circulatory diseases (e.g. arteriosclerosis, atherosclerosis, aortitis syndrome, polyarteritis nodosa, and myocardosis);
xxvi) collagen diseases (e.g. scleroderma, Wegener's granuloma, and Sjogren's syndrome);
xxvii) adiposis;
xxviii) eosinophilic fasciitis;
xxix) periodontal diseases (e.g. damage to gingiva, periodontium, alveolar bone or substantia ossea dentis);
xxx) nephrotic syndrome (e.g. glomerulonephritis);
xxxi) male pattern alopecia, alopecia senile;
xxxii) muscular dystrophy;
xxxiii) pyoderma and Sezary syndrome;
xxxiv) chromosome abnormality-associated diseases (e.g. Down's syndrome);
xxxv) Addison's disease;
xxxvi) active oxygen-mediated diseases [e.g. organ injury (e.g. ischemic circulation disorders of organs (e.g. heart, liver, kidney, digestive tract, etc.) associated with preservation, transplantation, or ischemic diseases (e.g. thrombosis, cardial infarction, etc.));
xxxvii) intestinal diseases (e.g. endotoxin shock, pseudomembranous colitis, and drug- or radiation-induced colitis);
xxxviii) renal diseases (e.g. ischemic acute renal insufficiency, chronic renal failure);
xxxix) pulmonary diseases (e.g. toxicosis caused by pulmonary oxygen or drugs (e.g. paracort, bleomycin, etc.), lung cancer, and pulmonary emphysema);
xxxx) ocular diseases (e.g. cataracta, iron-storage disease (siderosis bulbi), retinitis, pigmentosa, senile plaques, vitreous scarring, corneal alkali burn);
xxxxi) dermatitis (e.g. erythema multiforme, linear immunoglobulin A bullous dermatitis, cement dermatitis); and
xxxxii) other diseases (e.g. gingivitis, periodontitis, sepsis, pancreatitis, and diseases caused by environmental pollution (e.g. air pollution), aging, carcinogen, metastasis of carcinoma, and hypobaropathy)];
xxxxiii) diseases caused by histamine release or leukotriene C4 release; restenosis of coronary artery following angioplasty and prevention of postsurgical adhesions;
xxxxiv) autoimmune diseases and inflammatory conditions (e.g., primary mucosal edema, autoimmune atrophic gastritis, premature menopause, male sterility, juvenile diabetes mellitus, pemphigus vulgaris, pemphigoid, sympathetic ophthalmitis, lens-induced uveitis, idiopathic leukopenia, active chronic hepatitis, idiopathic cirrhosis, discoid lupus erythematosus, autoimmune orchitis, arthritis (e.g. arthritis deformans), or polychondritis);
xxxxv) Human Immunodeficiency Virus (HIV) infection, AIDS;
xxxxvi) allergic conjunctivitis;
xxxxvii) hypertrophic cicatrix and keloid due to trauma, burn, or surgery.

In addition, the tricyclic macrolides like e.g. sirolimus have liver regenerating activity and/or activities of stimulating hypertrophy and hyperplasia of hepatocytes. Therefore, the pharmaceutical composition of the present invention is useful for increasing the effect of the therapy and/or prophylaxis of liver diseases [e.g. immunogenic diseases (e.g. chronic autoimmune liver diseases such as autoimmune hepatic diseases, primary biliary cirrhosis or sclerosing cholangitis), partial liver resection, acute liver necrosis (e.g. necrosis caused by toxins, viral hepatitis, shock, or anoxia), hepatitis B, non-A non-B hepatitis, hepatocirrhosis, and hepatic failure (e.g. fulminant hepatitis, late-onset hepatitis and “acute-on-chronic” liver failure (acute liver failure on chronic liver diseases))].

Furthermore, a composition of the present invention is useful for increasing the effect of the prevention and/or treatment of various diseases because of the useful pharmacological activity of the tricyclic macrolides, such as augmenting activity of chemotherapeutic effect, activity of cytomegalovirus infection, anti-inflammatory activity, inhibiting activity against peptidyl-prolyl isomerase or rotamase, antimalarial activity, antitumor activity and so on. In addition, sirolimus may be used for the treatment of Huntington's disease

In one aspect, the present invention relates to a pharmaceutical composition in particulate form comprising sirolimus together with one or more pharmaceutically acceptable excipient, wherein the composition upon oral administration to a mammal in need thereof exhibits an AUC/AUC_{Control} value of at least about 1.3, the AUC values being determined under similar conditions. The composition used as a control is given in the same dosage and is a commercially available sirolimus composition intended for oral administration. In the present context, the control composition is Rapamune® tablets.

In specific embodiments, the AUC/AUC_{Control} value is at least about 1.5 such as about 1.75 or more, about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more or about 5.0 or more, the AUC values being determined under similar conditions.

After oral administration of a pharmaceutical composition according to the present invention it is contemplated that the plasma concentration versus time profile show an extended period of time in which the plasma concentration is maintained within the therapeutic window (i.e. the plasma concentration leads to a therapeutic effect) without leading to serious unwanted side effects. Thus, a reduction in peak concentration is also observed. Accordingly, the invention relates to a pharmaceutical composition in particulate form comprising sirolimus or a derivative or analogue thereof together with one or more pharmaceutically acceptable excipient, wherein the composition upon oral administration to a mammal in need thereof release sirolimus or a derivative or analogue thereof in a controlled manner and exhibits a Cₘₐₓ that is at the most about 80% of that of Cₘₐₓ for Rapamune® tablets such as, e.g., at the most about 75%, at the most about 70%, at the most about 65%, at the most about 60%, at the most about 55%, at the most about 50%, at the most about 45% or at the most about 40%.

In the present context the terms controlled release and modified release are intended to be equivalent terms covering any type of release of sirolimus from a composition of the invention that is appropriate to obtain a specific therapeutic or prophylactic response after administration to a subject. A person skilled in the art knows how controlled release/modified release differs from the release of plain tablets or capsules. The terms “release in a controlled manner” or “release in a modified manner” have the same meaning as stated above.

The terms controlled release/modified release include slow release (that results in a lower Cₘₐₓ and later tₘₐₓ, but t_{1/2} is unchanged), extended release (that results in a lower Cₘₐₓ, later tₘₐₓ, but apparent t_{1/2} is longer); delayed release (that result in an unchanged Cₘₐₓ, but lag time and, accordingly, tₘₐₓ is delayed, and t_{1/2} is unchanged) as well as pulsatile release, burst release, sustained release, prolonged release, chrono-optimized release, fast release (to obtain an enhanced onset of action) etc. Included in the terms is also e.g. utilization of specific conditions within the body e.g. different enzymes or pH changes in order to control the release of the drug substance.

To be more specific, after oral administration to a mammal, including a human, of a pharmaceutical composition according to the present invention containing a dose of 5 mg sirolimus, sirolimus is released in a controlled manner and will exhibit a Cₘₐₓ that is at the most about 30 ng/ml such as, e.g. at the most about 25 ng/ml or at the most about 20 ng/ml.

However, a reduction in peak concentration may not lead to a decrease in therapeutic effect. Accordingly, the present invention also relates to a pharmaceutical composition, wherein W₅₀ is at least about 2 hours, such as, e.g., at least about 3 hours, at least about 4 hours, at least about 5 hours, at least about 6 hours, at least about 7 hours, at least about 8 hours, at least about 9 hours or at least about 10 hours. Furthermore or moreover, a composition according to the invention has a C_{diff}=[Cₘₐₓ−C (t=12 hours)] that is less than that of Rapamune® tablets under the same conditions. If C_{diff} for Rapamune® tablets is set to 100 then C_{diff} of a composition according to the invention is normally about 95 or less such as, e.g., about 90 or less, about 85 or less, about 80 or less, about 75 or less, about 70 or less, about 65 or less, about 60 or less, about 55 or less, about 50 or less, about 45 or less or about 40 or less.

Thus, in one embodiment of the invention it is contemplated that pharmaceutical compositions according to the invention exhibit surprisingly higher bioavailability compared to commercially available formulations such as Rapamune®. In fact the bioavailability of sirolimus can according to the invention be increased by over 200% compared with the said commercially available products.

More specifically, after oral administration to a mammal, including a human, of a pharmaceutical composition of the invention containing 5 mg of sirolimus, sirolimus is released in a controlled manner and exhibits a C_{diff} of about 20 ng/ml or less such as, e.g., about 15 ng/ml or less, about 13 ng/ml or less, about 10 ng/ml or less, or about 5 ng/ml or less.

A pharmaceutical composition according to the invention releases sirolimus in a controlled manner in order to extend the therapeutic action of sirolimus. In one aspect the release may be pH dependant, i.e. the release predominantly takes place after passage of the stomach. Such a pH dependent release is mainly provided by means of enteric coating material as described herein. The release may also be pH independent, e.g. by providing the composition with a controlled release coating such as, e.g. a cellulose based coating like e.g. ethylcellulose. A combination may of course also be employed.

In general, the change in bioavailability and/or the changes in other bioavailability related parameters are normally determined by in vivo studies in a suitable animal model testing the compositions in question together with e.g. Rapamune® or a similar commercially available sirolimus-containing product. The use of a dog model for establishing evidence of the bioavailability of certain formulations is general practice in the pharmaceutical industry.

The studies relevant for sirolimus are non-randomized, cross-over studies, where each dog is it's own control. Four dogs, and four treatments are normally applied. As no iv injections are given, the bioavailabilities obtained are relative.

Further it is also contemplated that the need for simultaneous food intake in order to secure a sufficient uptake of sirolimus is significantly reduced or even completely abolished.

Thus, in specific embodiments the pharmaceutical compositions according to the invention can provide significant higher bioavailability of sirolimus, which may reduce the daily intake of sirolimus, and reduce or abolish the need for administration in connection with food intake, which provide for a higher degree of freedom for the recipient of the pharmaceutical compositions, and consequently the patients acceptance and/or compliance may be significantly improved. Furthermore, the compositions are contemplated to provide a significant reduction in side effects, especially side effect related to a high peak concentration (such as, e.g., vomiting and nausea) and provide for an extended release of sirolimus leading to a better therapy.

As mentioned above, one of the major challenges with respect to formulation of sirolimus compositions is to avoid an adverse food effect. In general, sirolimus is much better absorbed when it is administered orally together with food. A great variation in bioavailability is therefore seen following administration with or without food. This dependency makes it difficult to give precise guidelines as to how large a dose that should be administered and, furthermore, it requires information to the patient about the dosing regime. The present invention aims at providing compositions wherein the adverse food effect is reduced. Thus, the present invention provides a composition, which does not exhibit a significant adverse food effect after administration of the composition to a mammal in need of such a treatment as evidenced by a value of (AUC_{fed}/AUC_{fasted}) of at least about 0.85 with a lower 90% confidence limit of at least 0.75.

More specifically, in specific embodiments a pharmaceutical composition according to the invention can have a value of (AUC_{fed}/AUC_{fasted}) of about 0.9 or more such as, e.g., about 0.95 or more, about 0.97 or more or about 1 or more such as, e.g., up to about 1.1 or up to about 1.2.

A further advantage of a composition of the present invention is the possibility of obtaining an effective therapeutic response with a decreased dosage compared to traditional oral treatment. Accordingly, upon oral administration to a mammal in need thereof a pharmaceutical composition according to the invention releases sirolimus or an analogue thereof in a controlled manner and—in a specific embodiment—the composition can be essentially bioequivalent with Rapamune® or a similar commercially available sirolimus-containing product when administered in a dose that is at the about most about 85% w/w such as, e.g., at the most about 80% w/w, at the most about 75%, at the most about 70% w/w, at the most about 65% w/w, at the most about 60% w/w, at the most about 55% w/w or at the most about 50% w/w of the dose of sirolimus administered in the form of Rapamune® or a similar commercially available sirolimus-containing product.

Parameters often used in bioequivalence studies are tₘₐₓ, cₘₐₓ, AUC_{0-infinity}, AUC₀₋ₜ. Other relevant parameters may be W₅₀, W₇₅ and/or MRT. Accordingly, at least one of these parameters may be applied when determining whether bioequivalence is present. Furthermore, in the present context, two compositions are regarded as bioequivalent if value of the parameter used is within 80-125% of that of Rapamune® or a similar commercially available sirolimus-containing product used in the test.

In the present context “tₘₐₓ” denotes the time to reach the maximal plasma concentration (cₘₐₓ) after administration; AUC_{0-infinity} denotes the area under the plasma concentration versus time curve from time 0 to infinity; AUC₀₋ₜ denotes the area under the plasma concentration versus time curve from time 0 to time t; W₅₀ denotes the time where the plasma concentration is 50% or more of Cₘₐₓ; W₇₅ denotes the time where the plasma concentration is 75% or more of Cₘₐₓ; and MRT denotes mean residence time for sirolimus (and/or a derivative and/or an analogue thereof).

Two other main disadvantages associated with treatment or prophylaxis with sirolimus is the relative high incidence of gastrointestinal side effects and a relatively high inter-individual variation. It is envisaged that a composition according to the invention will lead to a reduction in unwanted side effects, especially gastrointestinal related side effects. The reduction may be in terms of reduced frequency or in terms of severity. The side effects in question include e.g. vomiting, nausea, diarrhea, constipation, abdominal pain, etc. In one aspect the invention concerns a pharmaceutical composition in particulate form comprising sirolimus or an analogue thereof together with one or more pharmaceutically acceptable excipient, wherein the composition upon oral administration to a mammal in need thereof releases sirolimus or an analogue thereof in a controlled manner and reduces gastro-intestinal side effects compared to those of Rapamune® administered under the same conditions and in a dose that provides an equivalent therapeutic effect.

Increasing the bioavailability, the Area Under the Curve, will normally reduce the intra- and inter-variability related to absorption of a drug substance. This is particularly true; whenever the low and impaired bioavailability is a consequence of poor water solubility. It is contemplated that compositions according to the invention will provide CV's (CV=coefficient of variation) on Area under Curve data that are significantly smaller than with Rapamune® and like products.

As mentioned hereinbefore, one of the basic features of the present invention is that for certain embodiments it is possible to obtain an improvement in the bioavailability by oral administration of a composition of the present invention. Normally, a low bioavailability of a drug substance after oral administration is a barrier for design of a controlled or modified release composition of the drug substance due to the fact that it is almost impossible to obtain effective drug levels over a prolonged period of time. However, with the present technology it is possible to obtain an acceptable bioavailability and thereby possible to design controlled, modified or delayed release compositions.

Sirolimus is extensively metabolized by the CYP3A4 isoenzyme in the gut wall and liver. Accordingly, a suitable controlled release composition may be a composition that is designed to release sirolimus in a delayed manner so as to avoid or reduce the CYP3A4 metabolism in the upper gastrointestinal tract.

Delayed release is mainly brought about by some kind of enteric coating. Whereas semipermeable coating will show some kind of delayed release, it does not preciously enough “delay” release. Additionally it requires a certain amount of time to release the content. The coating sought for this invention, may be a pH dependant coating. This type of coating is very resistant to release of drug until a certain pH is reached. Within very few 1/10'th of pH, the film alters properties and becomes permeable. Examples of pH-sensitive polymers, which are relatively insoluble and impermeable at the pH of the stomach, but which are more soluble and permeable at the pH of the small intestine and colon include, but not limited to polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

The release of the active substance from a composition having a delayed release coating could also be an enzymatic reaction, if for example Zein or mono/di-glyceride mixtures are employed as coating material.

Upon oral administration to a mammal, including a human, in need thereof, a controlled release pharmaceutical composition according to the present invention and designed for extended release releases sirolimus in such a manner that a plasma concentration of at least about 5 ng/ml such as, e.g., at least about 7.5 ng/ml or at least about 10 ng/ml for a time period of at least about 24 hours is obtained. In a specific aspect of the invention the difference between the peak plasma concentration and plasma concentration measured 24 hours after administration is at the most about 20 ng/ml such as, e.g., at the most about 10 ng/ml, at the most about 7.5 ng/ml or at the most about 5 ng/ml.

pH-sensitive polymers of specific interest include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Increasing the bioavailability, the Area Under the Curve, will normally reduce the intra- and inter-variability related to absorption of a drug substance. This is particularly true; whenever the low and impaired bioavailability is a consequence of poor water solubility. It is contemplated that compositions according to the invention will provide CV's on Area under Curve data that are significantly smaller than with Rapamune® and like products.

Furthermore, it is envisaged that a pharmaceutical composition comprising sirolimus together with one or more pharmaceutically acceptable excipient—and wherein the composition upon oral administration to a mammal in need thereof releases sirolimus or an analogue thereof in a controlled manner (dependent on the design of the composition, this may be a pH-dependant or a pH-independent manner)—reduces inter- and/or intra-individual variations compared to those of Rapamune® administered under the same conditions and in a dose that provides an equivalent therapeutic effect.

In a specific aspect, the invention provides a pharmaceutical composition or a solid dosage form that releases sirolimus and/or a derivative or analogue thereof relatively fast but extended so as to enable a relatively fast onset of therapeutic effect and a long maintenance of the therapeutic effect. Accordingly, the invention relates to a pharmaceutical composition in particulate form comprising sirolimus and/or an analogue thereof together with one or more pharmaceutically acceptable excipient, wherein the composition upon oral administration to a mammal in need thereof in a controlled manner releases at least about 50% w/w of the total amount of sirolimus and/or an analogue thereof within about 24 hours, such as, e.g., within about 22 hours, within about 20 hours, within about 18 hours, within about 15 hours or within about 12 hours.

In a further embodiment at the most about 60% w/w such as, e.g., at the most 62% w/w, at the most about 65% w/w or at the most about 70% w/w sirolimus is released 15 hours after oral administration to a mammal of a composition according to the invention or, alternatively, when tested in a suitable in vitro dissolution test, 15 hours after start of such a test.

More specifically, upon oral administration to a mammal in need thereof a composition according to the invention releases at least about 50% w/w of the total amount of sirolimus and/or an analogue thereof within about 10 hours such as, e.g., within about 8 hours, within about 6 hours, within about 4 hours or within about 3 hours.

In another embodiment, upon oral administration to a mammal in need thereof, a pharmaceutical composition according to the invention releases at least 80% w/w after about 0.5 hours or more such as, e.g., after about 0.75 hours or more, about 1 hour or more, about 2 hours or more, about 3 hours or more, about 4 hours or more or about 5 hours or more; or alternatively, when tested in a suitable in vitro dissolution test releases at least 80% w/w after about 0.5 hours or more such as, e.g., after about 0.75 hours or more, about 1 hour or more, about 2 hours or more, about 3 hours or more, about 4 hours or more or about 5 hours or more after start of the test.

In a further embodiment, upon oral administration to a mammal in need thereof a pharmaceutical composition according to the invention releases at least about 55% w/w such as, e.g., about 60% w/w or more, about 65% w/w or more, about 70% w/w or more, about 75% w/w or more or about 80% w/w or more of the total amount of sirolimus and/or an analogue thereof within about 24 hours such as, e.g., within about 22 hours, within about 20 hours, within about 18 hours, within about 15 hours within about 12 hours, within about 10 hours, within 8 hours or within about 6 hours.

Furthermore or alternatively, at least about 50% w/w of the total amount of sirolimus and/or an analogue thereof is released within 24 hours such as, e.g., within about 22 hours, within about 20 hours, within about 18 hours, within about 15 hours or within about 12 hours, when tested in an in vitro dissolution test and employing a dissolution medium comprising a buffer having pH 7.5. Guidance for a suitable dissolution test is described in the Examples herein, but variations with respect to the specific method employed and the ingredients contained in the dissolution medium etc. are within the scope of the present invention. A person skilled in the art will know how to carry out a suitable dissolution test e.g. with guidance from USP, Ph.Eur. and the like. Suitable conditions for the in vitro dissolution test are employing USP dissolution test (paddle method) and a buffer pH 7.5 containing 2.5% SDS and 1 g/mL of pancreatin as dissolution medium.

In other embodiments, the following conditions are fulfilled with respect to in vitro dissolution test:

i) at least about 50% w/w of the total amount of sirolimus or an analogue thereof is released within about 10 hours such as, e.g., within about 8 hours, within about 6 hours, within about 4 hours, within about 3 hours or within about 2 hours, when tested in an in vitro dissolution test and employing a dissolution medium comprising a buffer having pH 7.5,
ii) at least about 50% w/w of the total amount of sirolimus or an analogue thereof is released within about 1.5 hours such as, e.g., within about 1 hour, within about 0.75 hours, within about 0.5 hours or within about 20 minutes, when tested in an in vitro dissolution test and employing a dissolution medium comprising a buffer having pH 7.5,
iii) at least about 55% w/w such as, e.g., about 60% w/w or more, about 65% w/w or more, about 70% w/w or more, about 75% w/w or more or about 80% w/w or more of the total amount of sirolimus or an analogue thereof is released within about 15 hours such as, e.g., within about 12 hours, within about 10 hours, within 8 hours or within about 6 hours, when tested in an in vitro dissolution test and employing a dissolution medium comprising a buffer having pH 7.5,
iv) at least about 55% w/w such as, e.g., about 60% w/w or more, about 65% w/w or more, about 70% w/w or more, about 75% w/w or more or about 80% w/w or more of the total amount of sirolimus or an analogue thereof is released within about 5 hours such as, e.g., within about 4 hours, within about 3 hours, within about 2 hours, within about 1 hours or within about 30 minutes, when tested in an in vitro dissolution test and employing a dissolution medium comprising a buffer having pH 7.5, and/or
v) at least about 20% w/w such as, e.g., at least about 25% w/w, at least about 30% w/w, at least about 35% w/w or at least about 40% w/w of the total amount of sirolimus or an analogue thereof is released within the first 3 hours such as, e.g., within the first 2 hours or within the first hour when tested in an in vitro dissolution test and employing a dissolution medium comprising a buffer having pH 7.5.

In an interesting embodiment, the composition is designed to have a delayed release of sirolimus and/or an analogue thereof. Therefore, the invention also includes a pharmaceutical composition in particulate form comprising sirolimus and/or an analogue thereof together with one or more pharmaceutically acceptable excipient, wherein the composition upon oral administration to a mammal in need thereof has a delayed release of sirolimus and/or an analogue thereof so that at the most 10% w/w such as, e.g., at the most about 7.5% w/w or at the most about 5% w/w of the total amount of sirolimus or an analogue thereof is released within the first two hours such as, e.g., within the first hour after administration.

In other embodiments, the following conditions are fulfilled with respect to in vitro dissolution test performed under acidic conditions:

i) at the most about 30% w/w such as, e.g., at the most about 25% w/w, at the most about 20% w/w, at the most about 15% w/w or at the most about 10% w/w of sirolimus or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5,
ii) at the most about 10% w/w such as, e.g., at the most about 7.5% w/w, at the most about 5% w/w or at the most about 2.5% w/w of sirolimus or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5
iii) at the most about 60% w/w such as, e.g., at the most about 50% w/w, at the most about 40% w/w or at the most about 30% w/w of sirolimus or an analogue thereof is released within 15 hours such as, e.g., within about 12 hours, when tested in an in vitro dissolution test employing a dissolution medium having a pH of at the most about 4.5 such as, e.g. at the most about 4.0, at the most about 3.5, at the most about 3, at the most about 2 or at the most about 1.5,
iv) at the most about 40% w/w such as, e.g., at the most about 30% w/w, at the most about 25% w/w or at the most about 20% w/w of sirolimus or an analogue thereof is released within 6 hours when tested in an in vitro dissolution test employing a dissolution medium having a pH of at the most about 4.5 such as, e.g. at the most about 4.0, at the most about 3.5, at the most about 3, at the most about 2 or at the most about 1.5, and/or
v) at the most about 30% w/w such as, e.g., at the most about 25% w/w, at the most about 20% w/w or at the most about 15% w/w of sirolimus or an analogue thereof is released within 4 hours when tested in an in vitro dissolution test employing a dissolution medium having a pH of at the most about 4.5 such as, e.g. at the most about 4.0, at the most about 3.5, at the most about 3, at the most about 2 or at the most about 1.5.

Apart from sirolimus, a composition of the invention may also comprise a further therapeutically, prophylactically and/or diagnostically active substance. Notably combinations of sirolimus with at least one of the following active substances are of interest: Substances that are indicated for use in connection with organ transplantation such as, e.g., steroids, calcineurin inhibitors and/or anti-proliferative agents. Specific examples include prednisone, prednisolone, methylprednisone, cyclosporin, mycophenolate, azathioprine, tacrolimus, everolimus, mycophenolate sodium, and FTY720 (Novartis).

The pharmaceutical compositions may be prepared by any convenient method such as, e.g. granulation, mixing, spray drying etc. A particularly useful method is the method described in WO 03/004001. Herein is described a process for the preparation of particulate material by a controlled agglomeration method, i.e. a method, which enables a controlled growth in particle size. The method involves spraying a first composition comprising e.g. sirolimus and a carrier (in the present context the term vehicle is applied), which has been melted, onto a second solid carrier medium. Normally, the meltable carrier has a melting point of at least 5° C. but lower than the melting point of sirolimus. The melting point of the carrier may be in the range of 10° C. to 150° C., such as, e.g., in the range of 30° C. to 100° C. or in the range of 40° C. to 50° C. is most preferred. In the present context, the term vehicle is employed covering a suitable selection of carriers, namely those carriers that has a melting point of 80° C. or less and wherein the solubility of sirolimus is at least 0.5% w/w at a temperature corresponding to the melting point of the vehicle, Such vehicles have been found to be particularly useful in order to achieve the objects of the present invention.

It is within the skills of the average practioner to select a suitable carrier being pharmaceutical acceptable, capable of dispersing or at least partly dissolving sirolimus and having a melting point in the desired range using general knowledge and routine experimentation. Suitable candidate for carriers are described in WO 03/004001, which is herein incorporated by reference.

In the present context, suitable carriers are e.g. those mentioned as vehicles including an oil or an oily-like material (as discussed later herein) as well as those disclosed in WO 03/004001, provided that the above-mentioned properties with respect to melting point and solubility of sirolimus in the vehicle are achieved. An advantage of using the controlled agglomeration method described in WO 03/004001 is that it is possible to apply a relatively large amount of a melt to a particulate material without having an undesirable growth in particle size. Accordingly, in one embodiment of the invention, the particulate material of a pharmaceutical composition has a geometric weight mean diameter d_{gw} of ≧10 μm such as, e.g. ≧20 μm, from about 20 to about 2000, from about 30 to about 2000, from about 50 to about 2000, from about 60 to about 2000, from about 75 to about 2000 such as, e.g. from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.

### Pharmaceutically Acceptable Excipients

A composition according to the invention comprises one or more pharmaceutically acceptable excipients. In order to avoid any confusion between the terms “vehicle” and “pharmaceutically acceptable excipient”, it should be mentioned that a vehicle may of course also contain one or more pharmaceutically acceptable excipients and is normally composed of such ingredients. However, in order to qualify as a vehicle, the requirements mentioned above with respect to melting point and solubility of sirolimus must be fulfilled. Normally, the vehicle containing sirolimus is added to a solid composition comprising one or more pharmaceutically acceptable excipients in order to enable the pharmaceutical composition to be made. The vehicle containing sirolimus will normally not in itself have the finish and acceptance of a patient to constitute a final composition ready for therapeutic use.

In the present context the terms “pharmaceutically acceptable excipient” are intended to denote any material, which is inert in the sense that it substantially does not have any therapeutic and/or prophylactic effect per se. Such an excipient may be added with the purpose of making it possible to obtain a pharmaceutical, cosmetic and/or foodstuff composition, which have acceptable technical properties.

Examples of suitable excipients for use in a composition or solid dosage form according to the invention include fillers, diluents, disintegrants, binders, lubricants etc. or mixture thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples on suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the second composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in a composition or solid dosage form of the invention are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g. stabilising agents. The concentration of an antioxidant and/or a stabilizing agent in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance.

Examples of surfactants are given in the following.

Suitable excipients for use in a composition or a solid dosage form according to the invention are surfactants such as, e.g., hydrophobic and/or hydrophilic surfactants as those disclosed in WO 00/50007 in the name of Lipocine, Inc. Examples on suitable surfactants are
i) polyethoxylated fatty acids such as, e.g. fatty acid mono- or diesters of polyethylene glycol or mixtures thereof such as, e.g. mono- or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30, PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000,
ii) polyethylene glycol glycerol fatty acid esters, i.e. esters like the above-mentioned but in the form of glyceryl esters of the individual fatty acids;
iii) glycerol, propylene glycol, ethylene glycol, PEG or sorbitol esters with e.g. vegetable oils like e.g. hydrogenated castor oil, almond oil, palm kernel oil, castor oil, apricot kernel oil, olive oil, peanut oil, hydrogenated palm kernel oil and the like,
iv) polyglycerized fatty acids like e.g. polyglycerol stearate, polyglycerol oleate, polyglycerol ricinoleate, polyglycerol linoleate,
v) propylene glycol fatty acid esters such as, e.g. propylene glycol monolaurate, propylene glycol ricinoleate and the like,
vi) mono- and diglycerides like e.g. glyceryl monooleate, glyceryl dioleae, glyceryl mono- and/or dioleate, glyceryl caprylate, glyceryl caprate etc.;
vii) sterol and sterol derivatives;
viii) polyethylene glycol sorbitan fatty acid esters (PEG-sorbitan fatty acid esters) such as esters of PEG with the various molecular weights indicated above, and the various Tween® series;
ix) polyethylene glycol alkyl ethers such as, e.g. PEG oleyl ether and PEG lauryl ether;
x) sugar esters like e.g. sucrose monopalmitate and sucrose monolaurate;
xi) polyethylene glycol alkyl phenols like e.g. the Triton® X or N series; xii) polyoxyethylene-polyoxypropylene block copolymers such as, e.g., the Pluronic® series, the Synperonic® series, Emkalyx®, Lutrol®, Supronic® etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407;
xiii) sorbitan fatty acid esters like the Span® series or Ariacel® series such as, e.g. sorbinan monolaurate, sorbitan monopalmitate, sorbitan monooleate, sorbitan monostearate etc.;
xiv) lower alcohol fatty acid esters like e.g. oleate, isopropyl myristate, isopropyl palmitate etc.;
xv) ionic surfactants including cationic, anionic and zwitterionic surfactants such as, e.g. fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0, 1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% w/w or from about 30 to about 50% w/w.

In a specific aspect of the invention, the at least one of the one or more pharmaceutically acceptable excipient is selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

Such materials are is especially useful as a sorption material for oily materials in pharmaceuticals, cosmetics and/or foodstuff. In a specific embodiment, the material is used as a sorption material for oily materials in pharmaceuticals. The material that has the ability to function as a sorption material for oily materials is also denoted “oil sorption material”. Furthermore, in the present context the term “sorption” is used to denote “absorption” as well as “adsorption”. It should be understood that whenever one of the terms is used it is intended to cover the phenomenon absorption as well as adsorption.

Notably, the pharmaceutically acceptable excipient may comprise a silica acid or a derivative or salt thereof such as, e.g., silicon dioxide or a polymer thereof as a pharmaceutically acceptable excipient. Dependent on the quality employed a silicon dioxide may be a lubricant or it may be an oil sorption material. Qualities fulfilling the latter function seem to be most important.

In a specific embodiment, a composition or solid dosage form according to invention comprises a pharmaceutically acceptable excipient that is a silicon dioxide product that has properties corresponding to Aeroperl® (available from Degussa, Frankfurt, Germany).

As it appears from the examples herein, a very suitable material is Aeroperl® 300 (including materials with properties like or corresponding to those of Aeroperl® 300).

Use of an oil sorption material in compositions or dosage forms according to the invention is very advantageous for the preparation of pharmaceutical, cosmetic, nutritional and/or food compositions, wherein the composition comprises oil or an oily-like material. One of the advantages is that is it possible to incorporate a relatively large amount of oil and oily-like material and still have a material that is solid. Thus, it is possible to prepare solid compositions with a relatively high load of oily materials by use of an oil sorption material according to the invention. Within the pharmaceutical field it is an advantage to be able to incorporate a relatively large amount of an oil or an oily-like material in a solid composition especially in those situation where the active substance does not have suitable properties with respect to water solubility (e.g. poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, delayed, sustained and/or pulsed delivery of the active substance. Thus, in a specific embodiment it is used in the preparation of pharmaceutical compositions.

The oil sorption material for use in the processing into solid compositions normally absorbs about 5% w/w or more, such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more of an oil or an oily material and is still a solid material.

An important aspect of the invention is compositions or solid dosage forms comprising a hydrophilic, lipophilic, hydrophobic and/or amphiphilic material as a vehicle (see below)

### Vehicles

In the present context the term “vehicle” is used in a very broad sense including oils, waxes, semi-solid materials and materials that normally are used as solvents (such as organic solvents) or co-solvents within the pharmaceutical industry, and the term also includes therapeutically and/or prophylactically active substances that are in liquid form at ambient temperature; furthermore the term includes emulsions like e.g. microemulsions and nanoemulsions and suspensions. The vehicles that can be absorbed will normally be liquid at ambient or elevated temperature (for practical reasons the max. temperature is about 250° C.). They may be hydrophilic, lipophilic, hydrophobic and/or amphiphilic materials.

However, the oily materials that are suitable for use in the present context are substances or materials, which have a melting point of at least about 0° C. and at the most about 80° C. and, furthermore, the solubility of sirolimus is at least 0.5% w/w at a temperature corresponding to the melting point of the vehicle. The vehicle may be composed of only one substance or it may be a mixture of substances provided that the overall properties of the vehicle are in accordance with the requirements mentioned above.

In specific embodiments of the invention, the vehicle has a melting point of about 5° C. or more such as, e.g., about 10° C. or more, about 15° C. or more, about 20° C. or more or about 25° C. or more.

In further embodiments of the invention, the vehicle has a melting point of at least about 25° C. such as, e.g., at least about 30° C. at least about 35° C. or at least about 40° C. For practical reasons, the melting point may normally not be too high, thus, the oily material normally has a melting point of at the most about 80° C. If the melting point is higher a relatively high temperature may promote e.g. oxidation or other kind of degradation of an active substance in those cases where e.g. a therapeutically and/or prophylactically active substance is included.

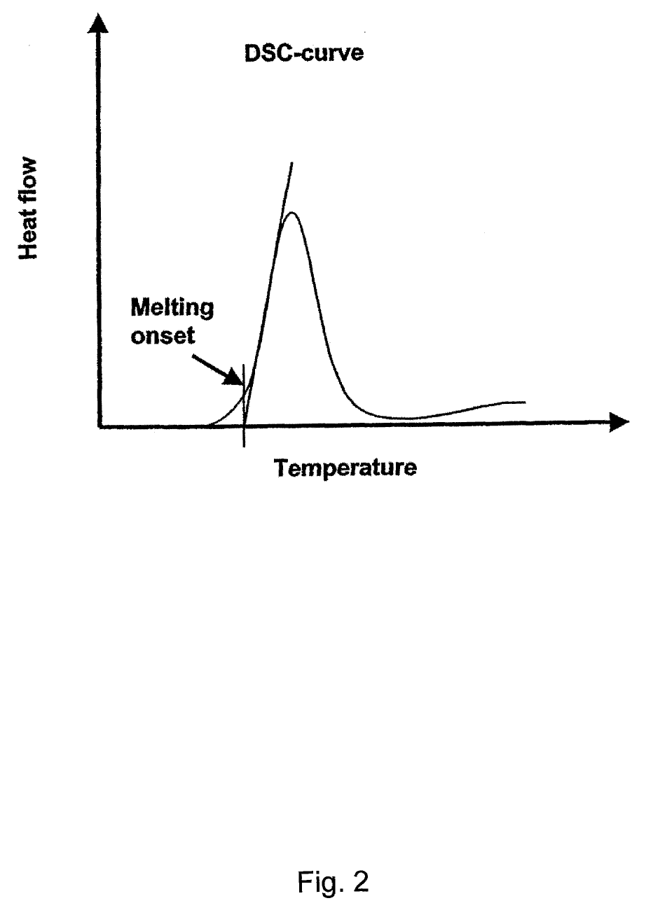

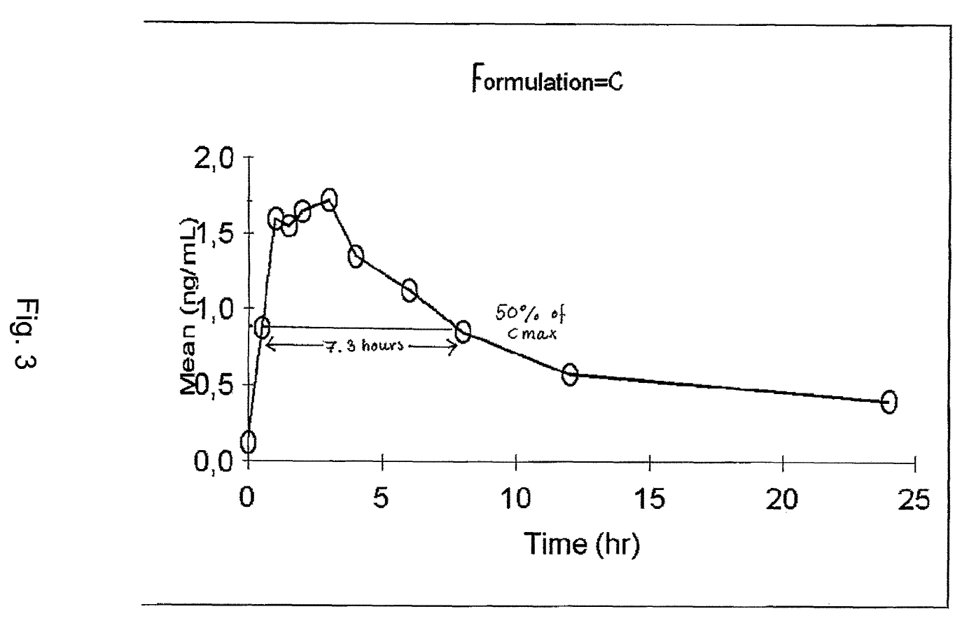

In the present context, the melting point is determined by DSC (Differential Scanning Calorimetry). The melting point is determined as the temperature at which the linear increase of the DSC curve intersects the temperature axis (see FIG. 2 for further details).

Interesting vehicles are generally substances, which are used in the manufacture of pharmaceuticals as so-called melt binders or solid solvents (in the form of solid dosage form), or as co-solvents or ingredients in pharmaceuticals for topical use.

It may be hydrophilic, hydrophobic and/or have surface-active properties. In general hydrophilic and/or hydrophobic materials are suitable for use in the manufacture of a pharmaceutical composition comprising a therapeutically and/or prophylactically active substance that has a relatively low aqueous solubility and/or when the release of the active substance from the pharmaceutical composition is designed to be immediate or non-modified. Hydrophobic oily materials, on the other hand, are normally used in the manufacture of a modified release pharmaceutical composition. The above-given considerations are simplified to illustrate general principles, but there are many cases where other combinations of oily materials and other purposes are relevant and, therefore, the examples above should not in any way limit the invention.

Typically, a suitable hydrophilic material for use as a vehicle (or a vehicle component) in the present context is selected from the group consisting of: polyether glycols such as, e.g., polyethylene glycols, polypropylene glycols; polyoxyethylenes; polyoxypropylenes; poloxamers and mixtures thereof, or it may be selected from the group consisting of: xylitol, sorbitol, potassium sodium tartrate, sucrose tribehenate, glucose, rhamnose, lactitol, behenic acid, hydroquinon monomethyl ether, sodium acetate, ethyl fumarate, myristic acid, citric acid, Gelucire 50/13, other Gelucire types such as, e.g., Gelucire 44/14 etc., Gelucire 50/10, Gelucire 62/05, Sucro-ester 7, Sucro-ester 11, Sucro-ester 15, maltose, mannitol and mixtures thereof.

A suitable hydrophobic material for use as a vehicle (or a vehicle component) in the present context may be selected from the group consisting of: straight chain saturated hydrocarbons, sorbitan esters, paraffins; fats and oils such as e.g., cacao butter, beef tallow, lard, polyether glycol esters; higher fatty acid such as, e.g. stearic acid, myristic acid, palmitic acid, higher alcohols such as, e.g., cetanol, stearyl alcohol, low melting point waxes such as, e.g., glyceryl monostearate, glyceryl monooleate, hydrogenated tallow, myristyl alcohol, stearyl alcohol, substituted and/or unsubstituted monoglycerides, substituted and/or unsubstituted diglycerides, substituted and/or unsubstituted triglycerides, yellow beeswax, white beeswax, carnauba wax, castor wax, japan wax, acetylate monoglycerides; NVP polymers, PVP polymers, acrylic polymers, or a mixture thereof. Some of these materials may not in themselves fulfill the above-mentioned criteria to qualify as a vehicle, but can then be mixed with other material(s) to obtain a vehicle that overall fulfills the criteria.

In an interesting embodiment, the vehicle comprises a polyethylene glycol having an average molecular weight in a range of from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000. In certain situations polyethylene glycol may be employed with a molecular weight from about 35,000 to about 100,000.

In another interesting embodiment, the vehicle comprises a polyethylene oxide having a molecular weight of from about 2,000 to about 7,000,000 such as, e.g. from about 2,000 to about 100,000, from about 5,000 to about 75,000, from about 10,000 to about 60,000, from about 15,000 to about 50,000, from about 20,000 to about 40,000, from about 100,000 to about 7,000,000 such as, e.g., from about 100,000 to about 1,000,000, from about 100,000 to about 600,000, from about 100,000 to about 400,000 or from about 100,000 to about 300,000.

In another embodiment, the vehicle comprises a poloxamer such as, e.g. Poloxamer 188, Poloxamer 237, Poloxamer 338 or Poloxamer 407 or other block copolymers of ethylene oxide and propylene oxide such as the Pluronic® and/or Tetronic® series. Suitable block copolymers of the Pluronic® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include Pluronic® F38, P65, P68LF, P75, F77, P84, P85, F87, F88, F98, P103, P104, P105, F108, P123, F123, F127, 10R8, 17R8, 25R5, 25R8 etc. Suitable block copolymers of the Tetronic® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

The vehicle may also contain a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

As mentioned above, the vehicle may of course comprise a mixture of different materials such as, e.g., a mixture of hydrophilic and/or hydrophobic materials.

Other suitable vehicles comprise solvents or semi-solid excipients like, e.g. propylene glycol, polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

Normally, a pharmaceutical composition or a solid dosage form according to the invention has a concentration of the oily material in the composition of about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more. In specific embodiments the concentration of the oily material in a composition or solid dosage form of the invention is in a range from about 20% to about 80% W/W such as, e.g., from about 25% to about 75% w/w.

One of the advantages is that is it possible to incorporate a relatively large amount of a vehicle and still end up with a composition that is solid. Thus, it is possible to prepare solid compositions with a relatively high load of a vehicle. Within the pharmaceutical field it is an advantage to be able to incorporate a relatively large amount of a vehicle in a solid composition especially in those situation where the active substance does not have suitable properties with respect to water solubility (e.g. poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, delayed, sustained and/or pulsed delivery of the active substance.

A further advantage is that the particulate material obtained is a free-flowing powder and therefore readily processable into e.g. solid dosage forms such as tablets, capsules or sachets. Normally, the particulate material has properties that are suitable in order to manufacture tablets by direct compression without addition of large amounts of further additives. A suitable test for test the flowability of the particulate material is the method described in Ph.Eur. and measuring the flow rate of the material out of a funnel with a nozzle (orifice) diameter of 10.0 mm.

In an important embodiment of the invention, at least a part of sirolimus and/or an analogue thereof is present in the composition in the form of a solid dispersion including a molecular dispersion and a solid solution. Normally, 10% or more such as, e.g., 20% or more, 30% or more, 40% or more, 50% or more, 60% or more, 70% or more, 80% or more, 90% or more such as, e.g., 95% or more or about 100% w/w of sirolimus and/or an analogue thereof is present in the composition in the form of a solid dispersion.

A solid dispersion may be obtained in different ways e.g. by dissolving sirolimus in the vehicle at a temperature of at the most 80° C. and in a concentration that is below the solubility of sirolimus in the vehicle in question or by employing organic solvents or by dispersing or dissolving the active substance in another suitable medium (e.g. a material that is in liquid form at room temperature or at elevated temperatures).

### Description of a Solid Dispersion Based on Organic Solvents

Solid dispersions (solvent method) are prepared by dissolving a physical mixture of the active substance (e.g. a drug substance) and the carrier in a common organic solvent, followed by evaporation of the solvent. The carrier is often a hydrophilic polymer. Suitable organic solvents include pharmaceutical acceptable solvent in which the active substance is soluble such as methanol, ethanol, methylene chloride, chloroform, ethyl acetate, acetone or mixtures thereof.

Suitable water soluble carriers include polymers such as polyethylene glycol, poloxamers, polyoxyethylene stearates, poly-ε-caprolactone, polyvinylpyrrolidone (PVP), polyvinylpyrrolidone-polyvinylacetate copolymer PVP-PVA (Kollidon VA64), poly-methacrylic polymers (Eudragit RS, Eudragit RL, Eudragit NE, Eudragit E) and polyvinyl alcohol (PVA), hydroxypropyl cellulose (HPC), hydroxypropyl methyl cellulose (HPMC), methyl cellulose, and poly(ethylene oxide) (PEO).

Polymers containing acidic functional groups may be suitable for solid dispersions, which release the active substance in a preferred pH range providing acceptable absorption in the intestines. Such polymers may be one or more selected from the group comprising hydroxypropyl methylcellulose phtalate (HMPCP), polyvinyl acetate phtalate (PVAP), hydroxypropylmethylcellulose acetate succinate (HPMCAS), alginate, carbomer, carboxymethylcellulose, methacrylic acid copolymer (Eudragit L, Eudragit S), shellac, cellulose acetate phthalate (CAP), starch glycolate, polacrylin, methyl cellulose acetate phtalate, hydroxypropyulcellulose acetate phthalate, cellulose acetate terephtahalate, cellulose acetate isophthalate and cellulose acetate trimellitate.

In relations to amounts of the active substance and the polymer in the solid dispersion, the weight ratio of active substance to polymer may be in a range of from about 3:1 to about 1:20. However, narrower ranger of from about 3:1 to about 1:5, such as, e.g., from about 1:1 to about 1:3 or about may also be used.

The solid dispersion is preferably formed by spray drying techniques, controlled agglomeration, freeze-drying or coating on carrier particles or any other solvent removal process. The dried product contains the active substance present in the form of a solid dispersion including a molecular dispersion and a solid solution.

As an alternative to the use of organic solvents the drug and polymer may be co-grinded or extruded at elevated temperatures (melt extrusion).

The pharmaceutical compositions comprising sirolimus at least partly in form of a solid dispersion or solution may in principle be prepared using any suitable procedure for preparing pharmaceutical compositions known within the art.

Apart from using the organic solvent based method, solid dispersion or solid solutions of sirolimus and/or an analogue thereof may be obtained by dispersing and/or dissolving sirolimus in the vehicle e.g. used in the controlled agglomeration method. Stabilizing agents etc. may be added in order to ensure the stability of the solid dispersion/solution.

In another aspect, the invention relates to a method for the preparation of a pharmaceutical composition according to the invention. In general, any suitable method within the pharmaceutical field may be employed. However, in order to enable incorporation of a relatively high amount of a vehicle especially the method described in WO 03/004001 (by the same inventors) has proved satisfactory. Details concerning the method are given in the above-identified publication, which is hereby incorporated by reference as well as in the Examples herein. In short, the invention provide a process for preparing a particulate pharmaceutical material comprising sirolimus and/or an analogue thereof which method comprises spraying a first composition in liquid form, said composition comprising a carrier and having a melting point greater than 5° C. onto a second composition comprising a support, said second composition being in the fluidised state and having a temperature less than the melting point of the carrier. In principle the active substance may be present in the carrier composition and/or in the second composition. However, in those cases where sirolimus and/or an analogue thereof should be present in the composition at least partly as a solid dispersion, it is advantageous to incorporate or dissolve sirolimus and/or an analogue thereof in the carrier composition.

### Solid Dosage Forms

A pharmaceutical composition according to the invention is in particulate form and may be employed as such. However, in many cases it is more convenient to present the composition in the form of granules, pellets, microspheres, nanoparticles and the like or in the form of solid dosage forms including tablets, capsules and sachets and the like.

A solid dosage form according to the invention may be a single unit dosage form or it may in the form of a polydepot dosage form contain a multiplicity of individual units such as, e.g., pellets, beads and/or granules.

Normally, a pharmaceutical composition or a solid dosage form of the invention is intended for administration via the oral, buccal or sublingual administration route.

The invention also relates to the above-mentioned presentation form. Within the scope of the invention are compositions/solid dosage forms that are intended to release sirolimus and/or an analogue thereof in a fast release, a delayed release or modified release manner.

A solid dosage form according to the present invention comprises a pharmaceutical composition in particulate form as described above. The details and particulars disclosed under this main aspect of the invention apply mutatis mutandis to the other aspects of the invention. Accordingly, the properties with respect to bioavailability, changes in bioavailability parameters, reduction in adverse food effect as well as release of sirolimus and/or an analogue thereof etc. described and/or claimed herein for pharmaceutical compositions in particulate form are analogues for a solid dosage form according to the present invention.

Normally, the concentration of the pharmaceutical composition in particulate form (i.e. before manufacturing into a specific dosage form and, accordingly, before addition of specific pharmaceutically acceptable excipients that are necessary to obtain the specific dosage form) is in a range of from about 5 to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form. In an embodiment of the invention, the concentration of the pharmaceutical composition in particulate form is 50% w/w or more of the dosage form.

A solid dosage form according to the invention is obtained by processing the particulate material according to the invention by means of techniques well-known to a person skilled in the art. Normally, it involves further addition of one or more of the pharmaceutically acceptable excipients mentioned herein.

The composition or solid dosage form according to the invention may be designed to release sirolimus and/or a derivative and/or an analogue thereof in any suitable manner provided a suitable bioavailability is obtained. Thus, the active substance may be released relatively fast in order to obtain an enhanced on-set of action, it may be released so as to follow zero or first order kinetics or it may be released in a controlled or modified manner in order to obtain a predetermined pattern of release. Plain formulations are also within the scope of the present invention.

The composition or solid dosage form according to the invention may also be coated with a film coating, an enteric coating, a modified release coating, a protective coating, an anti-adhesive coating etc.

A solid dosage form according to the invention may also be coated in order to obtain suitable properties e.g. with respect to release of the active substance. The coating may be applied on single unit dosage forms (e.g. tablets, capsules) or it may be applied on a polydepot dosage form or on its individual units.

Suitable coating materials are e.g. methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, zein, calcium pectinate.

Plasticizers and other ingredients may be added in the coating material. The same or different active substance may also be added in the coating material.

In the following is given a more detailed description of interesting embodiments of the invention, i.e. embodiments wherein the solid dosage forms are designed to release sirolimus and/or an analogue thereof in a controlled manner. In the present context, the term “controlled manner” is intended to include all types of release which differ from the release obtained from plain tablets. Thus, the term includes so-called “controlled release”, “modified release”, “sustained release”, “pulsed release”, “prolonged release”, burst release”, “slow release”, “extended release”, as well as the terms “delayed release” and pH dependant release. However, a specific aspect of the invention relates to a delayed release composition or dosage form, which in this context is intended to denote a composition or dosage form that at the most releases 10% w/w of the active substance within the first 2 hours after administration and/or after start of a dissolution test employing a dissolution medium having a pH of at the most about 3.

### Types of Modified Release Systems

A first class includes matrix systems, in which sirolimus is embedded or dispersed in a matrix of another material that serves to retard the release of sirolimus into an aqueous environment (i.e., the luminal fluid of the GI tract). When sirolimus is dispersed in a matrix of this sort, release of the drug takes place principally from the surface of the matrix. Thus the drug is released from the surface of a device, which incorporates the matrix after it diffuses through the matrix or when the surface of the device erodes, exposing the drug. In some embodiments, both mechanisms can operate simultaneously. The matrix systems may be large, i.e., tablet sized (about 1 cm), or small (<0.3 cm). The system may be unitary (e.g., a bolus), may be divided by virtue of being composed of several sub-units (for example, several capsules which constitute a single dose) which are administered substantially simultaneously, or may comprise a plurality of particles, also denoted a multiparticulate. A multiparticulate can have numerous formulation applications. For example, a multiparticulate may be used as a powder for filling a capsule shell, or used per se for mixing with food to ease the intake.

In a specific embodiment, a matrix multiparticulate, comprises a plurality of sirolimus-containing particles, each particle comprising sirolimus and/or an analogue thereof e.g. in the form of a solid solution/dispersion with one or more excipients selected to form a matrix capable of controlling the dissolution rate of the sirolimus into an aqueous medium. The matrix materials useful for this embodiment are generally hydrophobic materials such as waxes, some cellulose derivatives, or other hydrophobic polymers. If needed, the matrix materials may optionally be formulated with hydrophobic materials, which can be used as binders or as enhancers. Matrix materials useful for the manufacture of these dosage forms such as: ethylcellulose, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble or hydrophilic binders or release modifying agents which can optionally be formulated into the matrix include hydrophilic polymers such as hydroxypropyl cellulose (HPC), hydroxypropyl methyl cellulose (HPMC), methyl cellulose, poly (N-vinyl-2-pyrrolidinone) (PVP), poly(ethylene oxide) (PEO), poly(vinyl alcohol) (PVA), xanthan gum, carrageenan, and other such natural and synthetic materials. In addition, materials, which function as release-modifying agents include water-soluble materials such as sugars or salts. Preferred water-soluble materials include lactose, sucrose, glucose, and mannitol, as well as hydrophillic polymers like e.g. HPC, HPMC, and PVP.

In a specific embodiment, a multiparticulate product is defined as being processed by controlled agglomeration. In this case sirolimus is dissolved or partly dissolved in a suitable meltable carrier (i.e. vehicle) and sprayed on carrier particles comprising the matrix substance.

Suitable meltable carriers (i.e vehicles) are mentioned previously herein.

Alternatively, sirolimus is dissolved in an organic solvent together with the matrix substance and spray dried or applied to carrier particles. Solvents typically employed in the process include acetone, ethanol, isopropanol, ethyl acetate, and mixtures of two or more (for further details reference is given to the paragraphs under the heading Description of a solid dispersion based on organic solvents).

Once formed, sirolimus matrix multiparticulates may be blended with compressible excipients such as lactose, microcrystalline cellulose, dicalcium phosphate, and the like and the blend compressed to form a tablet. Disintegrants such as sodium starch glycolate or cross-linked poly(vinyl pyrrolidone) are also usefully employed. Tablets prepared by this method disintegrate when placed in an aqueous medium (such as the GI tract), thereby exposing the multiparticulate matrix, which releases sirolimus there from.

A further embodiment of a matrix system has the form of a hydrophilic matrix tablet containing sirolimus and/or an analogue thereof (e.g. in the form of a solid dispersion) as a multiparticulate product and an amount of hydrophilic polymer sufficient to provide a useful degree of control over the sirolimus dissolution. Hydrophilic polymers useful for forming the matrix include hydroxypropylmethyl cellulose (HPMC), hydroxypropyl cellulose (HPC), poly (ethylene oxide), poly(vinyl alcohol), xanthan gum, carbomer, carrageenan, and zooglan. A preferred material is HPMC. Other similar hydrophilic polymers may also be employed. In use, the hydrophilic material is swollen by, and eventually dissolves in, water. The sirolimus is released both by diffusion from the matrix and by erosion of the matrix. The sirolimus dissolution rate of these hydrophilic matrix tablets may be controlled by the amount, molecular weight and gel strengths of the hydrophilic polymer employed. In general, using a greater amount of the hydrophilic polymer decreases the dissolution rate, as does using a higher molecular weight polymer. Using a lower molecular weight polymer normally increases the dissolution rate. A matrix tablet typically comprises about 20 to 90% by weight of sirolimus and about 80 to 10% by weight of polymer.

A preferred matrix tablet comprises, by weight, about 30% to about 80% solid dispersion containing sirolimus and/or an analogue thereof about 15% to about 35% matrix former (such as, e.g., HPMC), 0% to about 35% lactose, 0% to about 20% microcrystalline cellulose, and about 0.25% to about 2% lubricant (such as, e.g., magnesium stearate).

The matrix systems as a class often exhibit non-constant release of the drug from the matrix. This result may be a consequence of the diffusive mechanism of drug release, and modifications to the geometry of the dosage form can be used with advantage to make the release rate of the drug more constant.

A second class of sirolimus controlled-release dosage forms of this invention includes membrane-moderated or reservoir systems. In this class, a reservoir of sirolimus e.g. in a solid solution/dispersion as a multiparticulate product is surrounded by a rate-limiting membrane. The sirolimus traverses the membrane by mass transport mechanisms well known in the art, including but not limited to dissolution in the membrane followed by diffusion across the membrane or diffusion through liquid-filled pores within the membrane.

These individual reservoir system dosage forms may be large, as in the case of a tablet containing a single large reservoir, or multiparticulate, as in the case of a capsule or poly-depot tablets containing a plurality of reservoir particles, each individually coated with a membrane. The coating can be non-porous, yet permeable to sirolimus (for example sirolimus may diffuse directly through the membrane), or it may be porous. As with other embodiments of this invention, the particular mechanism of transport is not believed to be critical.

Sustained release coatings as known in the art may be employed to fabricate the membrane, especially polymer coatings, such as a cellulose ester or ether, an acrylic polymer, or a mixture of polymers. Preferred materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Wurster coater, or a rotary fluid bed coater.

If desired, the permeability of the coating may be adjusted by blending of two or more materials. A particularly useful process for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water-soluble membrane additives are leached out of the membrane, leaving pores, which facilitate release of the drug. The membrane coating can also be modified by the addition of plasticizers, as known in the art.

A particularly useful variation of the process for applying a membrane coating comprises dissolving the coating polymer in a mixture of solvents chosen such that as the coating dries, a phase inversion takes place in the applied coating solution, resulting in a membrane with a porous structure.

In general, a support for mechanically strengthening the membrane is not required.

The morphology of the membrane is not of critical importance so long as the permeability characteristics enumerated herein are met. The membrane can be amorphous or crystalline. It can have any category of morphology produced by any particular process and can be, for example, an interfacial-polymerized membrane (which comprises a thin rate-limiting skin on a porous support), a porous hydrophilic membrane, a porous hydrophobic membrane, a hydrogel membrane, an ionic membrane, and other such materials which are characterized by controlled permeability to sirolimus.

In one embodiment of the invention it is an aim to reduce the exposure of the upper GI tract to high concentrations of sirolimus. Accordingly, suitable dosage forms include those forms, which incorporate a specific delay before the onset of controlled release of sirolimus. An exemplary embodiment can be illustrated by a tablet (or a particulate material) comprising a core containing sirolimus coated with a first coating of a polymeric material of the type useful for sustained release of sirolimus and a second coating of the type useful for delaying release of drugs when the dosage form is ingested. The first coating is applied over and surrounds the tablet or individual particles. The second coating is applied over and surrounds the first coating.

A tablet can be prepared by techniques well known in the art and contains a therapeutically useful amount of sirolimus plus such excipients as are necessary to form the tablet by such techniques.

The first coating may be a sustained release coating as known in the art, especially polymer coatings, to fabricate the membrane, as previously discussed for reservoir systems, or it could be a controlled release matrix core, which are coated a second time with a delayed release material.

Materials useful for preparing the second coating on the tablet include polymers known in the art as enteric coatings for delayed-release of pharmaceuticals. These most commonly are pH-sensitive materials such as cellulose acetate phthalate, cellulose acetate trimellitate, hydroxypropyl methyl cellulose phthalate, poly (vinyl acetate phthalate), and acrylic copolymers such as Eudragit L-100 (Röhm Pharma) and related materials, as more fully detailed below under “Delayed Release”. The thickness of the delayed-release coating is adjusted to give the desired delay property. In general, thicker coatings are more resistant to erosion and, consequently, yield a longer and more effective delay. Preferred coatings range from about 30 μm in thickness to about 3 mm in thickness.

With a hydrophobic matrix material like Glyceryl monostearate, no delay coating is necessary. The tablet will only start to release sirolimus when it reaches an area of enzymatic degradation, more specifically after the duodenum.

When ingested, the twice-coated tablet passes through the stomach, where the second coating prevents release of the sirolimus under the acidic conditions prevalent there. When the tablet passes out of the stomach and into the small intestine, where the pH is higher, the second coating erodes or dissolves according to the physicochemical properties of the chosen material. Upon erosion or dissolution of the second coating, the first coating prevents immediate or rapid release of the sirolimus and modulates the release so as to prevent the production of high peak concentrations, thereby minimizing side-effects.

A further preferred embodiment comprises a multiparticulate wherein each particle is dual coated as described above for tablets, first with a polymer designed to yield sustained release of the sirolimus and then coated with a polymer designed to delay onset of release in the environment of the GI tract when the dosage form is ingested.

The rate of sirolimus release from the sustained-release-coated multiparticulates (i.e., the multiparticulates before they receive the delayed-release coating) and methods of modifying the coating are also controlled by the factors previously discussed for reservoir system sirolimus multiparticulates.

The second membrane or coating for dual coated multiparticulates is a delayed-release coating which is applied over the first sustained-release coating, as disclosed above for tablets, and may be formed from the same materials. It should be noted that the use of the so-called “enteric” materials to practice this embodiment differs significantly from their use to produce conventional enteric dosage forms. With conventional enteric forms, the object is to delay release of the drug until the dosage form has passed the stomach and then to deliver the dose in the duodenum. Dosing of sirolimus directly and completely to the duodenum may be undesirable, however, due to the side effects sought to be minimized or avoided by this invention. Therefore, if conventional enteric polymers are to be used to practice this embodiment, it may be necessary to apply them significantly more thickly than in conventional practice, in order to delay drug release until the dosage form reaches the lower GI tract. However, it is also possible to effect a sustained or controlled delivery of sirolimus after the delayed-release coating has dissolved or eroded, therefore the benefits of this embodiment may be realized with a proper combination of delayed-release character with sustained-release character, and the delayed-release part alone may or may not necessarily conform to USP enteric criteria. The thickness of the delayed-release coating is adjusted to give the desired delay property. In general, thicker coatings are more resistant to erosion and, consequently, yield a longer delay.

A first delayed release embodiment according to the invention is a “pH-dependent coated dosage form” such as, e.g., a tablet or a capsule. In the case of a tablet it comprises a tablet core comprising sirolimus e.g. in a solid solution/dispersion as a multiparticulate product, a controlled release matrix of e.g. HPMC, a disintegrant, a lubricant, and one or more pharmaceutical carriers, such core being coated with a material, preferably a polymer, which is substantially insoluble and impermeable at the pH of the stomach, and which is more soluble and permeable at the pH of the small intestine. Preferably, the coating polymer is substantially insoluble and impermeable at pH<5.0, and water-soluble at pH>5.0. The tablet core may be coated with an amount of polymer sufficient to assure that substantially no release of sirolimus from the dosage form occurs until the dosage form has exited the stomach and has resided in the small intestine for about 15 minutes or greater, preferably about 30 minutes or greater, thus assuring that minimal sirolimus is released in the duodenum. Mixtures of a pH-sensitive polymer with a water-insoluble polymer may also be employed. Tablets are coated with an amount of polymer comprising from about 10% to about 80% of the weight of the sirolimus-containing tablet core. Preferred tablets are coated with an amount of polymer comprising about 15% to about 50% of the weight of the sirolimus tablet core.

pH-sensitive polymers which are very insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

The delay time before release of sirolimus, after the “pH-dependent coated tablet” dosage form has exited the stomach, may be controlled by choice of the relative amounts of Eudragit-L® and Eudragit-S® in the coating, and by choice of the coating thickness. Eudragit-L® films dissolve above pH 6.0, and Eudragit-Se films dissolve above 7.0, and mixtures dissolve at intermediate pH's. Since the pH of the duodenum is approximately 6.0 and the pH of the colon is approximately 7.0, coatings composed of mixtures of Eudragit-L® and Eudragit-S® provide protection of the duodenum from sirolimus. If it is desired to delay release of sirolimus until the sirolimus-containing “pH-dependent coated tablet” has reached the colon, Eudragit-S® may be used as the coating material, as described by Dew et al. (Br. J. Clin. Pharmac. 14 (1982) 405-408). In order to delay the release of sirolimus for about 15 minutes or more, preferably 30 minutes or more, after the dosage form has exited the stomach, preferred coatings comprise from about 9:1 to about 1:9 Eudragit-L®/Eudragit-S®, more preferably from about 9:1 to about 1:4 Eudragit-L®/Eudragit-S®. The coating may comprise from about 3% to about 70% of the weight of the uncoated tablet core. Preferably, the coating comprises from about 5% to about 50% of the weight of the tablet core.

The invention is further illustrated in the following examples without limiting it thereto.

### Methods

### Determination of Weight Variation

The tablets prepared in the Examples herein were subject to a test for weight variation performed in accordance with Ph. Eur.

### Determination of Average Tablet Hardness

The tablets prepared in the Examples herein were subject to at test for tablet hardness employing Schleuniger Model 6D apparatus and performed in accordance with the general instructions for the apparatus.

### Determination of Disintegration Time

The time for a tablet to disintegrate, i.e. to decompose into particles or agglomerates, was determined in accordance with Ph. Eur.

Determination of Geometric Weight Mean Diameter d_{gw}

The geometric weight mean diameter was determined by employment of a method of laser diffraction dispersing the particulate material obtained (or the starting material) in air. The measurements were performed at 1 bar dispersive pressure in Sympatec Helos equipment, which records the distribution of the equivalent spherical diameter. This distribution is fitted to a log normal volume-size distribution.

When used herein, “geometric weight mean diameter” means the mean diameter of the log normal volume-size distribution.

### Determination of Dissolution Rate

The dissolution rate was determined by employment of USP paddle dissolution method at 37° C.

### EXAMPLES

For the preparation of a pharmaceutical composition in particulate form according to the invention the method described in WO 03/004001 (by the present inventors) has been employed. The method ensures a controlled agglomeration process, i.e. a strict control of the growth in particle size while at the same time it is possible to use a relatively large amount of an oily material.

### Examples on Sirolimus Formulation Based on Controlled Agglomeration

HPMC refers to Metolose 90 SH (type 2208) or Metolose 60 SH (type 2910) from ShinEtsu, available in different degree of polymerisation (viscosity, 3-100.000 cP) Either tablets, capsules or granules might be enteric coated with different types of polymers such as hydroxypropylmethylcellulose acetate succinate (Aqoat), cellulose acetate phthalate CAP, hydroxypropylmethylcellulose phtalate HPMCP or methacrylic acid copolymers such as Eudragit L30D, Eudragit 100/S, Eudragit 100/L

### Example 1

Immediate release tablet

| Substances | % | mg |
|---|---|---|
| Sirolimus | 0.50 | 1.00 |
| Lactose 200 mesh | 49.75 | 100.00 |
| PEG 6000 | 34.48 | 69.30 |
| Poloxamer 188 | 14.78 | 29.70 |
| Magnesium stearate | 0.50 | 1.01 |
| Total | 100.00 | 201.01 |

Sirolimus is dissolved in Polyethylene glycol 6000 and Poloxamer 188 (70:30 w/w ratio) at 70° C. The solution is sprayed on 250 g lactose in a fluid bed Strea-1. The granular product is sieved through sieve 0.7 mm and blended with magnesium stearate for 0.5 min in a Turbula mixer.

The mixture is compressed into 8 mm tablets with a strength of 1 mg (200 mg tablet with compound cup shaped.

Mean disintegration time: 20 min, Hardness: 45 N

### Example 2

Modified release polydepot capsule based on swelling
hydrocolloid matrix of hydroxypropylcellulose

| Substance | % | mg |
|---|---|---|
| Sirolimus | 0.50 | 1.00 |
| HPMC | 20.00 | 40.00 |
| Lactose 200 mesh | 30.00 | 60.00 |
| PEG 6000 | 34.65 | 69.30 |
| Poloxamer 188 | 14.85 | 29.70 |
| Total | 100.00 | 200.00 |

Sirolimus is dissolved in Polyethylene glycol 6000 and Poloxamer 188 (70:30 w/w ratio) at 70° C. The solution is sprayed on a mixture of 150 lactose and 100 g HPMC in a fluid bed Strea-1. The granular product is sieved through sieve 0.7 mm and filled into hard gelatine capsules (200 mg)

### Example 3

Modified release polydepot capsule based on swelling
hydrocolloid matrix of hydroxypropylcellulose

| Substance | % | mg |
|---|---|---|
| Sirolimus | 0.50 | 1.00 |
| HPMC 2910 3 cp | 20.00 | 40.00 |
| Lactose 200 mesh | 30.00 | 60.00 |
| Glyceryl monostearate | 49.50 | 99.00 |
| Total | 100.00 | 200.00 |

Sirolimus is dissolved in Glycerylmonostearate at 70° C. The solution is sprayed on a mixture of 150 lactose and 100 g HPMC in a fluid bed Strea-1. The granular product is sieved through sieve 0.7 mm and filled into hard gelatine capsules (200 mg)

### Example 4

Modified release matrix tablet based on swelling
hydrocolloid matrix of hydroxypropylcellulose

| Substance | % | mg |
|---|---|---|
| Sirolimus | 0.50 | 1.00 |
| HPMC | 19.90 | 40.00 |
| Lactose 200 mesh | 29.85 | 60.00 |
| PEG 6000 | 34.48 | 69.30 |
| Poloxamer 188 | 14.78 | 29.70 |
| Magnesium stearate | 0.50 | 1.01 |
| Total | 100.00 | 201.01 |

Sirolimus is dissolved in Polyethylene glycol 6000 and Poloxamer 188 (70:30 w/w ratio) at 70° C. The solution is sprayed on 250 g lactose in a fluid bed Strea-1. The granular product is sieved through sieve 0.7 mm and blended with HPMC and magnesium stearate for 0.5 min in a Turbula mixer.

The mixture is compressed into 8 mm tablets with a strength of 1 mg (200 mg tablet with compound cup shaped.

Mean disintegration time: 20 min, Hardness: 45 N

### Example 5

Modified release matrix tablet based on lipophilic
matrix of glyceryl monostearate

| Substance | % | mg |
|---|---|---|
| Sirolimus | 0.50 | 1.00 |
| Lactose 200 mesh | 49.75 | 100.00 |
| Glycerylmonostearate | 49.25 | 99.00 |
| Magnesium stearate | 0.50 | 1.01 |
| | 100.00 | 201.01 |

Sirolimus is dissolved in Glyceryl monostearate at 70° C. The solution is sprayed on 250 g lactose in a fluid bed Strea-1. The granular product is sieved through sieve 0.7 mm and blended with magnesium stearate for 0.5 min in a Turbula mixer. The mixture is compressed into 8 mm tablets with a strength of 1 mg (200 mg tablet with compound cup shape.

Mean disintegration time: 20 min, Hardness: 45 N

### Example 6

Modified release polydepot capsule based
on lipophilic matrix of glyceryl-monostearate

| Substance | % | mg |
|---|---|---|
| Sirolimus | 0.50 | 1.00 |
| Lactose 200 mesh | 49.75 | 100.00 |
| Glycerylmonostearate | 49.25 | 99.00 |
| Magnesium stearate | 0.50 | 1.01 |
| | 100.00 | 201.01 |

Sirolimus is dissolved in Glyceryl monostearate at 70° C. The solution is sprayed on 250 g lactose in a fluid bed Strea-1. The granular product is sieved through sieve 0.7 mm and filled into hard gelatine capsules (200 mg).

### Example 7

Modified release polydepot Tablet based on
lipophilic matrix of gelucire 44/14

| Substance | % | mg |
|---|---|---|
| Sirolimus | 0.50 | 1.00 |
| Aeroperl 300 | 49.75 | 100.00 |
| Gelucire 44/14 | 49.25 | 99.00 |
| Magnesium stearate | 0.50 | 1.01 |
| | 100.00 | 201.01 |

Sirolimus is dissolved in gelucire at 70° C. The solution is sprayed on 250 g aeroperl in a fluid bed Strea-1. The granular product is sieved through sieve 0.7 mm and filled into hard gelatine capsules (200 mg).

The granulate is compressed into 8 mm tablets with strength of 1 mg (tablet weight 200 mg). Tablets are cup shaped.

Mean disintegration time: 25 min, Hardness: 43 N

### Example 8

Immediate release tablet with fast onset and decreased
variability Formulations of sirolimus used in three
PK-studies in dogs compared to Rapamune 1 mg.
Batch RD1032-2T (Immediate release tablet)

| | | % | mg |
|---|---|---|---|
| API | Sirolimus | 0.62 | 1.00 |
| Carrier | Lactose 200 mesh | 49.75 | 80.00 |
| Vehicle | PEG 6000 | 34.39 | 55.30 |
| Vehicle | Poloxamer 188 | 14.74 | 23.70 |
| Lubricant | magnesium stearate | 0.50 | 0.80 |
| Sum | | 100.00 | 160.80 |

The formulation is prepared similar to Example 1.

1.25% sirolimus is dissolved in PEG6000/Poloxamer 188 (70:30 w/w) and sprayed on lactose 200 mesh (50% carrier of total). The granulate was mixed with Mg-stearate and compressed into tablets. 8 mm compound cup. Tablet hardness: 39 N. Disintegration time. 8 min.

### Example 9

Modified release composition with low variability
Batch RD1032-1K (CR-capsule formulation, swelling granules)

| | | % | mg |
|---|---|---|---|
| API | Sirolimus | 0.75 | 1.00 |
| Carrier | HPMC Pharmacoat | 20.00 | 26.67 |
| | 606 | | |
| Carrier | Lactose 200 mesh | 20.00 | 26.67 |
| Vehicle | Rylo MD50 | 59.25 | 79.00 |
| Sum | | 100.00 | 133.33 |

The formulation is prepared similar to Example 3.

1.25% sirolimus is dissolved in glyceryl monostearate (Rylo MD50) and sprayed on hydroxypropylmethylcellulose and lactose (1:1) (40% carriers of total). The sieved granulate was filled into capsules.

### Example 10

Modified release composition with low peak
Batch RD 1032-T1 (CR-tablet formulation based on swelling matrix)

| | | % | mg |
|---|---|---|---|
| API | Sirolimus | 0.44 | 1.00 |
| Carrier | HPMC Pharmacoat | 12.21 | 27.71 |
| | 606 | | |
| Carrier | Lactose 200 mesh | 12.21 | 27.71 |
| Vehicle | Rylo MD50 | 34.65 | 78.66 |
| Lubricant | Magnesium. stearate | 0.50 | 1.14 |
| Ekstragranular phase | Avicel PH200 | 40.00 | 90.80 |
| Sum | | 100.00 | 227.00 |

The granulate prepared according to Example 9 was mixed with Avicel PH200 as extragranular phase and a lubricant magnesium stearate.

The granulate was compressed into tablets. 8 mm compound cup. Tablet hardness: 37 N. Disintegration time. 8 min.

### Example 11

### In Vivo Studies in Beagle Dogs

In vivo studies with the purpose of determining the pharmacokinetic properties of the compositions of the present invention including parameters relative to the commercially available sirolimus tablet formulation, i.e. Rapamune®, was performed using Beagle dogs.

The experimental work was performed four Beagle dogs which was dosed with 1 mg of sirolimus after a overnight fast The studies were conducted as two open, non-randomised, cross-over studies. Each animal was its own control. Oral doses of sirolimus were administered after i.m Primperan® due to nausea being a side effect of sirolimus. Water ad libitum was allowed 5 hours post dosing. Each dog was dosed with the specified formulation of sirolimus without taking the weight of the dog into consideration.

Blood samples were collected at vena jugularis externa at the following points of time: Pre-dose, 0.5 1, 1.5, 2, 3, 4, 6, 8, 12 and 24 hours after dosing. 4 ml of blood were collected, mixed with EDTA, and the samples were protected from light and were frozen (−80° C.). The blood samples were analyzed and results were given in ng/mL.

The four product tested is A: Rapamune® 1 mg; B: a fast onset tablet formulation according to Example 8; C a slow capsule formulation according to Example 9 and; C: a slow onset tablet according to Example 10.

### Example 12

### Standard Deviation (SD) and Coefficient of Variation (CV)

Comparison of a commercial reference immediate release product and a fast onset product according to the invention (Example 8) for relevant pharmacokinetic parameters tested in fasted dogs as described in Example 11; N=8 (sum of 2 studies with 4 dogs in each group) (concentrations in ng/mL)

The advantage of using the coefficient of variation (CV=SD/Mean) is because this value is comparable across data sets with significant different means

| | | | | | |
|---|---|---|---|---|---|
| Reference A | | | | | |
| SD | 6.256 | 1.461 | 12.539 | 22.896 | 12.658 |
| CV % | 38.7 | 57.1 | 52 | 59.2 | 39 |
| Mean | | | | | |
| Test B | | | | | |
| SD | 4.98 | 0.697 | 3.02 | 3.759 | 9.02 |
| CV % | 28.1 | 29.3 | 20.4 | 16.9 | 27.5 |
| Percentage | 27% | 49% | 61% | 71% | 29% |
| decrease of | | | | | |
| CV % of test | | | | | |
| B compared | | | | | |
| with | | | | | |
| reference A | | | | | |

### Example 13

Standard deviation and coefficient of variation of a modified release formulation according to the invention tested according to Example 11; N=4

| Mean | | | | | |
|---|---|---|---|---|---|
| Test C | HL_Lambda_z | Cmax | AUClast | AUCINF_obs | AUC_% Extrap_pred |
| SD | 3.852 | 0.995 | 4.983 | 6.296 | 5.145 |
| CV % | 22.5 | 41 | 26.7 | 22.2 | 15.2 |
| Percentage | 42% | 28% | 48% | 62% | 61% |
| decrease of | | | | | |
| CV % of CR | | | | | |
| extended | | | | | |
| capsule (C) | | | | | |
| formulation | | | | | |
| compared with | | | | | |
| reference A | | | | | |

### Example 14

Test of fast onset determined by concentration at T=0.5 h and T=1 h between commercial reference immediate release product and a fast onset product according to the invention (Example 8) tested in dogs as describe in Example 11; N=4 Concentration at T=0.5 hour and T=1 hour

| | | | Percentage |
|---|---|---|---|
| | | | increase in |
| | (ng/mL) | | concentration of |
| mean | A reference | B test | formulation B |
| T = 0.5 h | 0.975 | 1.8125 | 86% |
| T = 1.0 h | 1.645 | 2.05 | 25% |

### Example 15

Test of Cdiff of modified release formulations (Examples 9 and 10) according to the invention and tested in dogs as describe in Example 11; N=4

Cdiff=[Cmax−C(t=12 hours)].

| | | % of |
|---|---|---|
| Rapamune ® | ng/mL | Rapamune |
| Mean Concentration t = 12 | | |
| hours (n = 8) | 0.80625 | |
| Cmax | 2.561 | |
| Cdiff | 1.75475 | |
| Modified release capsule | | |
| formulation of Example 9 | | |
| Mean Concentration t = 12 | | |
| hours (n = 4) | 0.766667 | |
| Cmax | 2.425 | |
| Cdiff | 1.658333 | 94.5 |
| Modified release tablet | | |
| formulation of Example 10 | | |
| Mean Concentration t = 12 | | |
| hours (n = 4) | 0.475 | |
| Cmax | 1.35 | |
| Cdiff | 0.875 | 49.9 |

### Example 16

Test of W₅₀ from modified release formulations (Examples 9 and 10) according to the invention and tested in dogs as describe in Example 11; N=4

W₅₀ denotes the time where the plasma concentration is 50% or more of Cₘₐₓ.

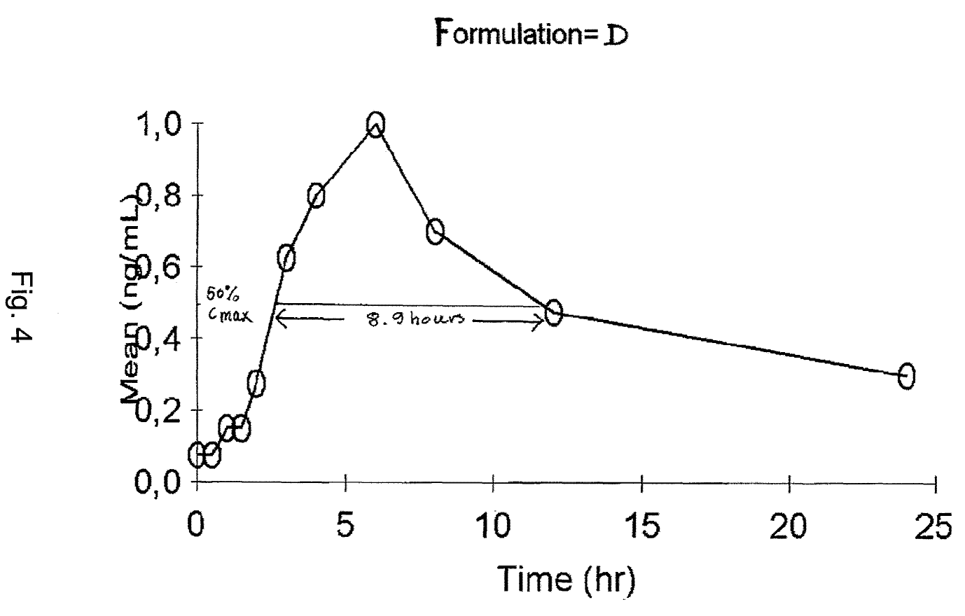

From FIG. 3 it appears that the time where the plasma concentration is above 50% of the maximal concentration is about 7.3 hours for the modified release capsule formulation according to Example 9. This indicates an extended effect of the formulation. Similarly for test formulation D, the modified release tablet of Example 10 comprising the same granular composition as C, the concentration is above 50% of the maximal concentration for at least 8.9 hours. Accordingly, the tablet has a more extended effect compared with the capsule.

### SPECIFIC EMBODIMENTS OF THE INVENTION

1. A pharmaceutical composition in particulate form comprising sirolimus or a derivative or analogue thereof together with one or more pharmaceutically acceptable excipient, wherein the composition upon oral administration to a mammal in need thereof releases sirolimus or an analogue thereof in a controlled manner and exhibits an AUC/AUC_{Control} value of at least about 1.3, the AUC values being determined under similar conditions.
2. A pharmaceutical composition according to item 1, wherein the AUC/AUC_{Control} value is at least about 1.5 such as about 1.75 or more, about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more or about 5.0 or more, the AUC values being determined under similar conditions.
3. A pharmaceutical composition in particulate form comprising sirolimus or a derivative or analogue thereof together with one or more pharmaceutically acceptable excipient, wherein the composition upon oral administration to a mammal in need thereof release sirolimus or a derivative or analogue thereof in a controlled manner and exhibits a Cₘₐₓ that is at the most about 80% of that of Cₘₐₓ for Rapamune® tablets such as, e.g., at the most about 75%, at the most about 70%, at the most about 65%, at the most about 60%, at the most about 55%, at the most about 50%, at the most about 45% or at the most about 40%.
4. A pharmaceutical composition in particulate form comprising sirolimus or a derivative or analogue thereof together with one or more pharmaceutically acceptable excipient, wherein the composition upon oral administration to a mammal in need thereof release sirolimus or a derivative or analogue thereof in a controlled manner and exhibits a W₅₀ that is about 2 hours or more such as, e.g., about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more or about 14 hours or more.
5. A pharmaceutical composition in particulate form comprising sirolimus or a derivative or analogue thereof together with one or more pharmaceutically acceptable excipient, wherein the composition upon oral administration to a mammal in need thereof release sirolimus or a derivative or analogue thereof in a controlled manner and exhibits a C_{diff} of 90 or less such as, e.g., about 85 or less, about 80 or less, about 75 or less, about 70 or less, about 65 or less, about 60 or less, about 55 or less, about 50 or less, about 45 or less or about 40 or less, when C_{diff}=[Cₘₐₓ−C (t=12 hours)] and C_{diff} for Rapamune® tablets is set to 100.
6. A pharmaceutical composition in particulate form comprising sirolimus or a derivative or analogue thereof together with one or more pharmaceutically acceptable excipient, wherein the composition upon oral administration to a mammal in need thereof releases sirolimus or an analogue thereof in a controlled manner and does not exhibit a significant adverse food effect as evidenced by a value of (AUC_{fed}/AUC_{fasted}) of at least about 0.85 with a lower 90% confidence limit of at least 0.75.
7. A pharmaceutical composition according to item 6, wherein the value of (AUC_{fed}/AUC_{fasted}) is about 0.9 or more such as, e.g., about 0.95 or more, about 0.97 or more or about 1 or more.
8. A pharmaceutical composition in particulate form comprising sirolimus together or an analogue thereof with one or more pharmaceutically acceptable excipient, wherein the composition upon oral administration to a mammal in need thereof releases sirolimus or an analogue thereof in a controlled manner and the composition being essentially bioequivalent with Rapamune® or a similar commercially available sirolimus-containing product when administered in a dose that is at the about most about 85% w/w of the dose of sirolimus administered in the form of Rapamune® or a similar commercially available sirolimus-containing product.
9. A pharmaceutical composition according to item 8, wherein the dose is at the most about 80% w/w such as, e.g., at the most about 75%, at the most about 70% w/w, at the most about 65% w/w, at the most about 60% w/w, at the most about 55% w/w or at the most about 50% w/w of the dose of sirolimus administered in the form of Rapamune® or a similar commercially available sirolimus-containing product.
10. A pharmaceutical composition according to item 8 or 9, wherein the bioequivalence is determined by means of at least one of the following parameters: tₘₐₓ, cₘₐₓ, AUC₀₋ₜ, AUC_{0-infinity}, W₅₀, W₇₅ and/or MRT.
11. A pharmaceutical composition in particulate form comprising sirolimus or an analogue thereof together with one or more pharmaceutically acceptable excipient, wherein the composition upon oral administration to a mammal in need thereof releases sirolimus or an analogue thereof in a controlled manner and reduces gastro-intestinal side effects compared to those of Rapamune® administered under the same conditions and in a dose that provides an equivalent therapeutic effect.
12. A pharmaceutical composition in particulate form comprising sirolimus or an analogue thereof together with one or more pharmaceutically acceptable excipient, wherein the composition upon oral administration to a mammal in need thereof releases sirolimus or an analogue thereof in a controlled manner and reduces inter- and/or intra-individual variations compared to those of Rapamune® administered under the same conditions and in a dose that provides an equivalent therapeutic effect.
13. A pharmaceutical composition in particulate form comprising sirolimus or an analogue thereof together with one or more pharmaceutically acceptable excipient, wherein the composition upon oral administration to a mammal in need thereof in a controlled manner releases at least about 50% w/w of the total amount of sirolimus or an analogue thereof within about 24 hours, such as, e.g., within about 22 hours, within about 20 hours, within about 18 hours, within about 15 hours or within about 12 hours.
14. A pharmaceutical composition according to item 13, wherein the composition upon oral administration to a mammal in need thereof releases at least about 50% w/w of the total amount of sirolimus or an analogue thereof within about 10 hours such as, e.g., within about 8 hours, within about 6 hours, within about 4 hours or within about 3 hours.
15. A pharmaceutical composition according to item 13 or 14, wherein the composition upon oral administration to a mammal in need thereof releases at least about 55% w/w such as, e.g., about 60% w/w or more, about 65% w/w or more, about 70% w/w or more, about 75% w/w or more or about 80% w/w or more of the total amount of sirolimus or an analogue thereof within about 24 hours, such as, e.g., within about 22 hours, within about 20 hours, within about 18 hours, within about 15 hours or within about 12 hours, within about 10 hours, within 8 hours or within about 6 hours.
16. A pharmaceutical composition according to item 13, wherein at least about 50% w/w of the total amount of sirolimus or an analogue thereof is released within about 24 hours, such as, e.g., within about 22 hours, within about 20 hours, within about 18 hours, within about 15 hours or within about 12 hours, when tested in an in vitro dissolution test and employing a dissolution medium comprising a buffer having pH 7.5.
17. A pharmaceutical composition according to any of items 13-16, wherein at least about 50% w/w of the total amount of sirolimus or an analogue thereof is released within about 10 hours such as, e.g., within about 8 hours, within about 6 hours, within about 4 hours, within about 3 hours or within about 2 hours, when tested in an in vitro dissolution test and employing a dissolution medium comprising a buffer having pH 7.5.
18. A pharmaceutical composition according to item 17, wherein at least about 50% w/w of the total amount of sirolimus or an analogue thereof is released within about 1.5 hours such as, e.g., within about 1 hour, within about 0.75 hours, within about 0.5 hours or within about 20 minutes, when tested in an in vitro dissolution test and employing a dissolution medium comprising a buffer having pH 7.5.
19. A pharmaceutical composition according to any of items 13-18, wherein at least about 55% w/w such as, e.g., about 60% w/w or more, about 65% w/w or more, about 70% w/w or more, about 75% w/w or more or about 80% w/w or more of the total amount of sirolimus or an analogue thereof is released within about 15 hours such as, e.g., within about 12 hours, within about 10 hours, within 8 hours or within about 6 hours, when tested in an in vitro dissolution test and employing a dissolution medium comprising a buffer having pH 7.5.
20. A pharmaceutical composition according to any of items 13-18, wherein at least about 55% w/w such as, e.g., about 60% w/w or more, about 65% w/w or more, about 70% w/w or more, about 75% w/w or more or about 80% w/w or more of the total amount of sirolimus or an analogue thereof is released within about 5 hours such as, e.g., within about 4 hours, within about 3 hours, within about 2 hours, within about 1 hours or within about 30 minutes, when tested in an in vitro dissolution test and employing a dissolution medium comprising a buffer having pH 7.5.
21. A pharmaceutical composition according to any of items 16-20, wherein the in vitro dissolution test is carried out employing USP dissolution test (paddle) and a buffer pH 7.5 containing 2.5% SDS and 1 g/mL of pancreatin as dissolution medium.
22. A pharmaceutical composition according to item 13, wherein at least about 20% w/w such as, e.g., at least about 25% w/w, at least about 30% w/w, at least about 35% w/w or at least about 40% w/w of the total amount of sirolimus or an analogue thereof is released within the first 3 hours such as, e.g., within the first 2 hours or within the first hour when tested in an in vitro dissolution test and employing a dissolution medium comprising a buffer having pH 7.5.
23. A pharmaceutical composition in particulate form comprising sirolimus or an analogue thereof together with one or more pharmaceutically acceptable excipient, wherein the composition upon oral administration to a mammal in need thereof has a delayed release of sirolimus or an analogue thereof so that at the most about 10% w/w such as, e.g., at the most about 7.5% w/w or at the most about 5% w/w of the total amount of sirolimus or an analogue thereof is released within the first two hours such as, e.g., within the first hour after administration.
24. A pharmaceutical composition according to item 23, wherein at the most about 30% w/w such as, e.g., at the most about 25% w/w, at the most about 20% w/w, at the most about 15% w/w or at the most about 10% w/w of sirolimus or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5.
25. A pharmaceutical composition according to item 23 or 24, wherein at the most about 10% w/w such as, e.g., at the most about 7.5% w/w, at the most about 5% w/w or at the most about 2.5% w/w of sirolimus or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5.
26. A pharmaceutical composition according to any of items 23-25, wherein at the most about 60% w/w such as, e.g., at the most about 50% w/w, at the most about 40% w/w or at the most about 30% w/w of sirolimus or an analogue thereof is released within 15 hours such as, e.g., within about 12 hours, when tested in an in vitro dissolution test employing a dissolution medium having a pH of at the most about 4.5 such as, e.g. at the most about 4.0, at the most about 3.5, at the most about 3, at the most about 2 or at the most about 1.5.
27. A pharmaceutical composition according to any of items 23-26, wherein at the most about 40% w/w such as, e.g., at the most about 30% w/w, at the most about 25% w/w or at the most about 20% w/w of sirolimus or an analogue thereof is released within 6 hours when tested in an in vitro dissolution test employing a dissolution medium having a pH of at the most about 4.5 such as, e.g. at the most about 4.0, at the most about 3.5, at the most about 3, at the most about 2 or at the most about 1.5.
28. A pharmaceutical composition according to any of items 23-27, wherein at the most about 30% w/w such as, e.g., at the most about 25% w/w, at the most about 20% w/w or at the most about 15% w/w of sirolimus or an analogue thereof is released within 4 hours when tested in an in vitro dissolution test employing a dissolution medium having a pH of at the most about 4.5 such as, e.g. at the most about 4.0, at the most about 3.5, at the most about 3, at the most about 2 or at the most about 1.5.
29. A pharmaceutical composition according to any of the preceding items, wherein the particulate material has a geometric weight mean diameter d_{gw} of ≧10 μm such as, e.g. >20 μm, from about 20 to about 2000, from about 30 to about 2000, from about 50 to about 2000, from about 60 to about 2000, from about 75 to about 2000 such as, e.g. from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.
30. A pharmaceutical composition according to any of the preceding items, wherein the one or more pharmaceutically acceptable excipient is selected from the group consisting of fillers, disintegrants, binders, diluents, lubricants and glidants.
31. A pharmaceutical composition according to any of the preceding items further comprising an pharmaceutically acceptable additive selected from the group consisting of flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents.
32. A pharmaceutical composition according to any of the preceding items wherein at least one of the one or more pharmaceutically acceptable excipient is selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.
33. A pharmaceutical composition according to any of the preceding items comprising a silica acid or a derivative or salt thereof.
34. A pharmaceutical composition any of the preceding items comprising silicon dioxide or a polymer thereof.
35. A pharmaceutical composition according to any of the preceding items comprising a silicon dioxide product that has properties corresponding to Aeroperl® 300 (available from Degussa, Frankfurt, Germany).
36. A pharmaceutical composition according to any of the preceding items comprising an oil or an oily-like material.
37. A pharmaceutical composition according to item 36, wherein the concentration of the oily material in the composition is about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more.
38. A pharmaceutical composition according to item 37, wherein the concentration of the oily material is in a range from about 20% to about 80% w/w such as, e.g., from about 25% to about 75% w/w.
39. A pharmaceutical composition according to any of the preceding items, wherein at least a part of sirolimus or an analogue thereof, is present in the form of a solid dispersion including a molecular dispersion and a solid solution.
40. A pharmaceutical composition according to item 39, wherein the solid dispersion is manufactured by dissolving at least a part of sirolimus or an analogue thereof in an organic solvent containing a material suitable for forming solid dispersions and subsequent removing the organic solvent e.g. by evaporation.
41. A pharmaceutical composition according to item 40, wherein the material suitable for forming solid dispersions is selected from the group consisting of cellulose derivatives including hydroxypropylmethylcellulose, NaCMC, PVP and PVA.
42. A pharmaceutical composition according to any of the preceding items having an acceptable flowability as determined according to the method described in Ph.Eur. measuring the flow rate of the material out of a funnel with a nozzle diameter of 10.0 mm.
43. A pharmaceutical composition according to any of the preceding items for use in the manufacture of granules, pellets, microspheres, nanoparticles.
44. A pharmaceutical composition according to any of the preceding items for use in the manufacture of a solid dosage form.
45. A pharmaceutical composition according to item 44, wherein the solid dosage form is intended for administration via the oral, buccal or sublingual administration route.
46. A pharmaceutical composition according to item 44 or 45 in the form of tablets, capsules or sachets.
47. A pharmaceutical composition according to any of the preceding items for use in the manufacture of tablets obtained by direct compression.
48. A solid dosage form comprising a pharmaceutical composition according to any of items 1-47.
49. A solid dosage form according to item 48, wherein the concentration of the pharmaceutical composition in particulate form is in a range of from about 5% to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form.
50. A solid dosage form according to item 48, wherein the concentration of the pharmaceutical composition in particulate form is 50% w/w or more of the dosage form.
51. A solid dosage form according to any of items 48-50, wherein the solid dosage form upon oral administration to a mammal in need thereof exhibits an AUC/AUC_{Control} value of at least about 1.3, the AUC values being determined under similar conditions.
52. A solid dosage form according to item 51, wherein the AUC/AUC_{Control} value is at least about 1.5 such as about 1.75 or more, about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more or about 5.0 or more, the AUC values being determined under similar conditions.
53. A solid dosage form according to any of items 48-53, wherein the solid dosage form releases sirolimus or an analogue thereof in a controlled manner and does not exhibit a significant adverse food effect as evidenced by a value of (AUC_{fed}/AUC_{fasted}) of at least about 0.85 with a lower 90% confidence limit of at least 0.75.
54. A solid dosage form according to item 53, wherein the value of (AUC_{fed}/AUC_{fasted}) is about 0.9 or more such as, e.g., about 0.95 or more, about 0.97 or more or about 1 or more.
55. A solid dosage form according to any of items 48-54, wherein the solid dosage form upon oral administration to a mammal in need thereof releases sirolimus or an analogue thereof in a controlled manner and the solid dosage form being essentially bioequivalent with Rapamune® or a similar commercially available sirolimus-containing product when administered in a dose that is at the most about 85% w/w of the dose of sirolimus administered in the form of Rapamune® or a similar commercially available sirolimus containing product.
56. A solid dosage form according to item 55, wherein the dose is at the most about 80% w/w such as, e.g., at the most about 75%, at the most about 70% w/w, at the most about 65% w/w, at the most about 60% w/w, at the most about 55% w/w or at the most about 50% w/w of the dose of sirolimus administered in the form of Rapamune® or a similar commercially available sirolimus-containing product.
57. A solid dosage form according to item 55 or 56, wherein the bioequivalence is determined by means of at least one of the following parameters: tₘₐₓ, cₘₐₓ, AUC₀₋ₜ, AUC_{0-infinity}, W₅₀, W₇₅ and/or MRT.
58. A solid dosage form comprising sirolimus or an analogue thereof together with one or more pharmaceutically acceptable excipient, wherein the solid dosage form upon oral administration to a mammal in need thereof releases sirolimus or an analogue thereof in a controlled manner and reduces gastro-intestinal side effects compared to those of Rapamune® administered under the same conditions and in a dose that provides an equivalent therapeutic effect.
59. A solid dosage form comprising sirolimus or an analogue thereof together with one or more pharmaceutically acceptable excipient, wherein the solid dosage form upon oral administration to a mammal in need thereof releases sirolimus or an analogue thereof in a controlled manner and reduces inter- and/or intra-individual variations compared to those of Rapamune® administered under the same conditions and in a dose that provides an equivalent therapeutic effect.
60. A solid dosage form according to any of items 48-59, wherein the solid dosage form upon oral administration to a mammal in need thereof releases at least about 50% w/w of the total amount of sirolimus or an analogue thereof within about 24 hours, such as, e.g., within about 22 hours, within about 20 hours, within about 18 hours, within about 15 hours or within about 12 hours.
61. A solid dosage form according to item 60, wherein the solid dosage form upon oral administration to a mammal in need thereof releases at least about 50% w/w of the total amount of sirolimus or an analogue thereof within about 10 hours such as, e.g., within about 8 hours, within about 6 hours, within about 4 hours or within about 3 hours.
62. A solid dosage form according to item 60 or 61, wherein the solid dosage form upon oral 10 administration to a mammal in need thereof releases at least about 55% w/w such as, e.g., about 60% w/w or more, about 65% w/w or more, about 70% w/w or more, about 75% w/w or more or about 80% w/w or more of the total amount of sirolimus or an analogue thereof within about 24 hours, such as, e.g., within about 22 hours, within about 20 hours, within about 18 hours, within about 15 hours, within about 12 hours, within about 10 hours, within 8 hours or within about 6 hours.
63. A solid dosage form according to item 60, wherein at least about 50% w/w of the total amount of sirolimus or an analogue thereof is released within 24 hours, such as, e.g., within about 22 hours, within about 20 hours, within about 18 hours, within about 15 hours or within about 12 hours, when tested in an in vitro dissolution test and employing a dissolution medium comprising a buffer having pH 7.5.
64. A solid dosage form according to any of items 60-63, wherein at least about 50% w/w of the total amount of sirolimus or an analogue thereof is released within about 10 hours such as, e.g., within about 8 hours, within about 6 hours, within about 4 hours, within about 3 hours or within about 2 hours, when tested in an in vitro dissolution test and employing a dissolution medium comprising a buffer having pH 7.5.
65. A solid dosage form according to item 64, wherein at least about 50% w/w of the total amount of sirolimus or an analogue thereof is released within about 1.5 hours such as, e.g., within about 1 hour, within about 0.75 hours, within about 0.5 hours or within about 20 minutes, when tested in an in vitro dissolution test and employing a dissolution medium comprising a buffer having pH 7.5.
66. A solid dosage form according to any of items 61-65, wherein at least about 55% w/w such as, e.g., about 60% w/w or more, about 65% w/w or more, about 70% w/w or more, about 75% w/w or more or about 80% w/w or more of the total amount of sirolimus or an analogue thereof is released within about 15 hours such as, e.g., within about 12 hours, within about 10 hours, within 8 hours or within about 6 hours, when tested in an in vitro dissolution test and employing a dissolution medium comprising a buffer having pH 7.5.
67. A solid dosage form according to any of items 61-66, wherein at least about 55% w/w such as, e.g., about 60% w/w or more, about 65% w/w or more, about 70% w/w or more, about 75% w/w or more or about 80% w/w or more of the total amount of sirolimus or an analogue thereof is released within about 5 hours such as, e.g., within about 4 hours, within about 3 hours, within about 2 hours, within about 1 hours or within about 30 minutes, when tested in an in vitro dissolution test and employing a dissolution medium comprising a buffer having pH 7.5.
68. A solid dosage form according to any of items 63-67, wherein the in vitro dissolution test is carried out employing USP dissolution test (paddle) and a buffer pH 7.5 containing 2.5% SDS and 1 g/mL of pancreatin as dissolution medium.
69. A solid dosage form according to item 60, wherein at least about 20% w/w such as, e.g., at least about 25% w/w, at least about 30% w/w, at least about 35% w/w or at least about 40% w/w of the total amount of sirolimus or an analogue thereof is released within the first 3 hours such as, e.g., within the first 2 hours or within the first hour when tested in an in vitro dissolution test and employing a dissolution medium comprising a buffer having pH 7.5.
70. A solid dosage form according to any of items 48-50, wherein the solid dosage form upon oral administration to a mammal in need thereof has a delayed release of sirolimus or an analogue thereof so that at the most about 10% w/w such as, e.g., at the most about 7.5% w/w or at the most about 5% w/w of the total amount of sirolimus or an analogue thereof is released within the first two hours such as, e.g., within the first hour after administration.
71. A solid dosage form according to item 70, wherein at the most about 30% w/w such as, e.g., at the most about 25% w/w, at the most about 20% w/w, at the most about 15% w/w or at the most about 10% w/w of sirolimus or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5.
72. A solid dosage form according to item 70 or 71, wherein at the most about 10% w/w such as, e.g., at the most about 7.5% w/w, at the most about 5% w/w or at the most about 2.5% w/w of sirolimus or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5.
73. A solid dosage form according to any of items 70-72, wherein at the most about 60% w/w such as, e.g., at the most about 50% w/w, at the most about 40% w/w or at the most about 30% w/w of sirolimus or an analogue thereof is released within 15 hours such as, e.g., within about 12 hours, when tested in an in vitro dissolution test employing a dissolution medium having a pH of at the most about 4.5 such as, e.g. at the most about 4.0, at the most about 3.5, at the most about 3, at the most about 2 or at the most about 1.5.
74. A solid dosage form according to any of items 70-73, wherein at the most about 40% w/w such as, e.g., at the most about 30% w/w, at the most about 25% w/w or at the most about 20% w/w of sirolimus or an analogue thereof is released within 6 hours when tested in an in vitro dissolution test employing a dissolution medium having a pH of at the most about 4.5 such as, e.g. at the most about 4.0, at the most about 3.5, at the most about 3, at the most about 2 or at the most about 1.5.
75. A solid dosage form according to any of items 70-74, wherein at the most about 30% w/w such as, e.g., at the most about 25% w/w, at the most about 20% w/w or at the most about 15% w/w of sirolimus or an analogue thereof is released within 4 hours when tested in an in vitro dissolution test employing a dissolution medium having a pH of at the most about 4.5 such as, e.g. at the most about 4.0, at the most about 3.5, at the most about 3, at the most about 2 or at the most about 1.5.
76. A solid dosage form according to any of items 48-75 comprising a multiplicity of individual units such as, e.g., pellets, beads and/or granules.
77. A solid dosage form according to any of items 48-76 in the form of tablets, capsules or sachets.
78. A solid dosage form according to item 77 in the form of a tablet.
79. A solid dosage form according to any of items 77-78, wherein the individual units or solid dosage form are coated with a coating selected from the group consisting of film coatings, modified release coatings, enteric coatings, protective coatings and anti-adhesive coatings for the purpose of releasing sirolimus in a controlled manner.
80. A solid dosage form according to any of items 48-79, wherein the amount of sirolimus or an analogue thereof contained therein corresponds to a daily dose thereof.
81. A solid dosage form according to any of items 48-80, wherein sirolimus is embedded in a matrix that releases sirolimus by diffusion.
82. A solid dosage form according to item 81, wherein the matrix remains substantially intact during the period of drug release.
83. A solid dosage form according to any of items 48-81, wherein sirolimus is embedded in a matrix that release sirolimus by eroding.
84. A solid dosage form according to any of items 48-80, wherein sirolimus is released from the dosage form by diffusion through a substantially water-insoluble coating.
85. A solid dosage form according to any of items 48-80 in the form of a polydepot dosage form, which—upon administration—disintegrates into a multiplicity of individual units from which sirolimus is released.

1. A pharmaceutical composition comprising sirolimus together with one or more pharmaceutically acceptable excipient, wherein the composition upon oral administration to a mammal releases sirolimus in a controlled manner and reduces inter- and/or intra-individual variations compared to those of Rapamune® administered under the same conditions.

2. A pharmaceutical composition according to claim 1 comprising a vehicle that has a melting point of 80° C. or less and wherein the solubility of sirolimus is at least 0.5% w/w at a temperature corresponding to the melting point of the vehicle, wherein the coefficient of variation (CV) of Cₘₐₓ and/or of AUC_{inf} after administration to six healthy fasting subjects is at the most 30%.

3. A pharmaceutical composition according to claim 1 comprising a vehicle that has a melting point of 80° C. or less and wherein the solubility of sirolimus is at least 0.5% w/w at a temperature corresponding to the melting point of the vehicle, wherein the coefficient of variation (CV) of Cₘₐₓ and/or of AUC_{inf} after administration to four healthy fasting dogs is at the most 30%.

4. A pharmaceutical composition according to claim 1, wherein the CV of AUC_{inf} is at the most 25%.

5. A pharmaceutical composition according to claim 1, wherein the ratio (CV_{Control}−CV)/CV_{Control}×100% is at least 20% and the of CV is the CV of Cₘₐₓ and/or of AUC_{inf}, and CV_{Control} is determined under similar conditions as CV using Rapamune® tablets as control.

6. A pharmaceutical composition according to claim 5, wherein the ratio is at least 25%.

7. A pharmaceutical composition according to claim 5, wherein the CV is CV of Cₘₐₓ.

8. A pharmaceutical composition according to claim 5, wherein the ratio is at least 30% such as, e.g., at least 35%, at least 40%, at least 45% or at least 50%.

9. A pharmaceutical composition according to claim 5, wherein the CV is CV of AUC_{inf}.

10. A pharmaceutical composition according to claim 2, wherein the vehicle comprises at least one of Rylo MD50, Gelucire 44/14, PEG such as PEG 6000, Poloxamer such as Poloxamer 188, Monomuls 90 L12 and Monomuls 90 35, and mixtures thereof.

11. A pharmaceutical composition according to claim 1 in solid form.

12. A pharmaceutical composition according to claim 1 in a solid dosage form including tablets.

13. A pharmaceutical composition according to claim 2, wherein the concentration of sirolimus in the vehicle at the most corresponds to the solubility of sirolimus in the vehicle at 70° C.

14. A pharmaceutical composition according to claim 2, wherein the concentration of sirolimus in the vehicle is at the most about 10% w/w such as at the most about 5% w/w, at the most about 4% w/w, at the most about 3% w/w, at the most about 2% w/w or at the most about 1% w/w.

15. A pharmaceutical composition according to claim 1, wherein the preparation of the composition involves a step, wherein sirolimus is dissolved in the vehicle at a temperature in a range of from about 50° C. to about 80° C.

16. A pharmaceutical composition according to claim 1 in the form of a solid dosage form comprising one or more multipla of 0.25 mg of sirolimus.

17. A pharmaceutical composition according to claim 1 comprising from about 0.25 mg to about 5 mg of sirolimus.

18. A pharmaceutical composition according to claim 1 comprising a dose of 0.75 mg, 1 mg, 1.2 mg, 1.5 mg or 2 mg of sirolimus.

19. A pharmaceutical composition according to claim 18 comprising from about 50% to about 80% of said dose.

20. A pharmaceutical composition according to claim 1, wherein the concentration of sirolimus in the composition is from about 0.05% to about 20% w/w such as, e.g., from about 0.05% to about 15% w/w, from about 0.05 to about 10% w/w, from about 0.1% to about 10% w/w.

21. A pharmaceutical composition according to claim 1, wherein the concentration of sirolimus in the composition is from about 0.05% to about 5% w/w, from about 0.1% to about 5% w/w, from about 0.1% to about 2.5% w/w, from about 0.5% to about 2.5% w/w, from about 1% to about 2.5% or 1% w/w or less.

22. A pharmaceutical composition according to claim 2, wherein the vehicle at the most constitute 60% w/w of the composition.

23. A pharmaceutical composition according to claim 2, wherein the vehicle at least constitute 20% w/w of the composition such as, e.g. at least about 30% w/w or at least about 40% w/w.

24. A pharmaceutical composition according to claim 1, wherein sirolimus is released in a manner to provide fast onset of action after administration to a subject.

25. A pharmaceutical composition according to claim 24, wherein T_{0.5h} is at least 50% of Tₘₐₓ such as, e.g., at least 60%, at least 65%, at least 70%, at least 75% or at least 80% of Tₘₐₓ and T_{0.5h} and Tₘₐₓ are determined as average values after administration to six healthy fasting subject or four healthy fasting dogs.

26. A pharmaceutical composition according to claim 24, wherein T₁ₕ is at least 80% of Tₘₐₓ such as, e.g., at least 85%, at least 90%, at least 95% of Tₘₐₓ and T_{0.5h} and Tₘₐₓ are determined as average values after administration to six healthy fasting subject or four healthy fasting dogs.

27. A pharmaceutical composition according to claim 24, wherein Tₘₐₓ is at the most 1.5 hours such as, e.g., 1.2 hours, 1.1 hours or 1 hours determined as an average of Tₘₐₓ after administration to six healthy fasting subjects.

28. A pharmaceutical composition according to claim 24, wherein Tₘₐₓ is at the most 1.5 hours such as, e.g., 1.2 hours, 1.1 hours or 1 hours as determined as an average of Tₘₐₓ after administration to our healthy fasting dogs.

29. A pharmaceutical composition according to claim 24, wherein Tₘₐₓ/T_{max, Control}×100% is at the most 70% such as, e.g., at the most 65%, at the most 60% or at the most 55%.

30. A pharmaceutical composition according to claim 24, wherein the composition essentially does not contain HPMC.

31. A pharmaceutical composition according to claim 30, wherein the composition does not contain HPMC.

32. A pharmaceutical composition according to claim 1, wherein at least 50% w/w of sirolimus is released within 24 hours when tested in an in vitro dissolution test according to USP.

33. A pharmaceutical composition according to claim 32, wherein the composition upon oral administration to a mammal releases sirolimus in a controlled manner and exhibits a Cₘₐₓ that is at the most about 80% of that of Cₘₐₓ for Rapamune® tablets such as, e.g., at the most about 75%, at the most about 70%, at the most about 65%, at the most about 60%, at the most about 55%, at the most about 50%, at the most about 45% or at the most about 40%.

34. A pharmaceutical composition according to claim 32, wherein the composition upon oral administration to a mammal in need thereof releases sirolimus thereof in a controlled manner and exhibits a W₅₀ that is about 2 hours or more such as, e.g., about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more or about 14 hours or more.

35. A pharmaceutical composition according to claim 32, wherein the composition upon oral administration to a mammal in need thereof releases sirolimus in a controlled manner and exhibits a C_{diff} of 90 or less such as, e.g., about 85 or less, about 80 or less, about 75 or less, about 70 or less, about 65 or less, about 60 or less, about 55 or less, about 50 or less, about 45 or less or about 40 or less, when C_{diff}=[Cₘₐₓ−C (t=12 hours)] and C_{diff} for Rapamune® tablets is set to 100.

36. A pharmaceutical composition according to claim 32, wherein the composition upon oral administration to a mammal in need thereof releases sirolimus in a controlled manner and exhibits an AUC/AUC_{Control} value of at least about 1.3, the AUC values being determined under similar conditions.

37. A pharmaceutical composition according to claim 36, wherein the AUC/AUC_{Control} value is at least about 1.5 such as about 1.75 or more, about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more or about 5.0 or more, the AUC values being determined under similar conditions.

38. A pharmaceutical composition according to claim 32, wherein the composition upon oral administration to a mammal in need thereof releases sirolimus in a controlled manner and reduces gastro-intestinal side effects compared to those of Rapamune®administered under the same conditions.

39. A pharmaceutical composition according to claim 32, wherein the composition upon oral administration to a mammal releases sirolimus in a controlled manner and does not exhibit a significant adverse food effect as evidenced by a value of (AUC_{fed}/AUC_{fasted}) of at least about 0.85 with a lower 90% confidence limit of at least 0.75.

40. A pharmaceutical composition according to claim 39, wherein the value of (AUC_{fed}/AUC_{fasted}) is about 0.9 or more such as, e.g., about 0.95 or more, about 0.97 or more or about 1 or more.

41. A pharmaceutical composition according to claim 32, wherein the composition upon oral administration to a mammal releases sirolimus in a controlled manner and the composition being essentially bioequivalent with Rapamune® or a similar commercially available sirolimus-containing product when administered in a dose that is at the about most about 85% w/w of the dose of sirolimus administered in the form of Rapamune® or a similar commercially available sirolimus-containing product.

42. A pharmaceutical composition according to claim 41, wherein the dose is at the most about 80% w/w such as, e.g., at the most about 75%, at the most about 70% w/w, at the most about 65% w/w, at the most about 60% w/w, at the most about 55% w/w or at the most about 50% w/w of the dose of sirolimus administered in the form of Rapamune® or a similar commercially available sirolimus-containing product.

43. A pharmaceutical composition according to claim 41, wherein the bioequivalence is determined by means of at least one of the following parameters: tₘₐₓ, cₘₐₓ, AUC₀₋ₜ, AUC_{0-infinity}, W₅₀, W₇₅ and/or MRT.

44. A pharmaceutical composition according to claim 2, wherein the vehicle comprises one or more hydrophilic, lipophilic, hydrophobic and/or amphiphilic materials.

45. A pharmaceutical composition according to claim 2, wherein at least a part of sirolimus or an analogue thereof, is present in the form of a solid dispersion including a molecular dispersion and a solid solution.

46. A pharmaceutical composition according to claim 45, wherein the solid dispersion is manufactured by dissolving at least a part of sirolimus or an analogue thereof in an organic solvent containing a material suitable for forming solid dispersions and subsequent removing the organic solvent e.g. by evaporation.

47. A pharmaceutical composition according to claim 45, wherein the material suitable for forming a solid dispersion is the vehicle.

48. A pharmaceutical composition according to claim 45, wherein the material suitable for forming solid dispersions is selected from the group consisting of cellulose derivatives including hydroxypropylmethylcellulose, NaCMC, PVP and PVA.

49. A method for the preparation of a pharmaceutical composition as defined in claim 1, the method comprising
i) dissolving or dispersing sirolimus in a vehicle at a temperature of from about 50° C. to about 80° C.,
ii) adding the mixture obtained in step i) to a composition in powder or particulate form comprising one or more pharmaceutically acceptable excipients,
iii) manufacturing the thus obtained powder into a pharmaceutical composition.

50. A method according to claim 49, wherein step ii) involves spraying the heated mixture obtained in step i) on a composition in powder or particulate form comprising one or more pharmaceutically acceptable excipients.
